(19) United States
(12) Patent Application Publication
(10) Pub. No.: US 2025/0361462 A1
(43) Pub. Date: Nov. 27, 2025

(54) FRAGRANCE COMPOSITIONS HAVING LONG-LASTING TRAIL PERFORMANCE

(71) Applicant: FIRMENICH SA, Satigny (CH)

(72) Inventors: Denis SHCHERBAKOV, Ewing, NJ (US); Addi FADEL FADEL, Metuchen, NJ (US); Nicholas O'LEARY, Pennington, NJ (US)

(73) Assignee: FIRMENICH SA, Satigny (CH)

(21) Appl. No.: 18/874,987

(22) PCT Filed: Jun. 21, 2023
(86) PCT No.: PCT/EP2023/066745

§ 371 Date: Dec. 13, 2024

Related U.S. Application Data
(60) Provisional application No. 63/354,535, filed on Jun. 22, 2022.

(30) Foreign Application Priority Data
Jul. 27, 2022 (EP) 22187312.8

(51) Int. Cl. C11B 9/00

(52) U.S. Cl. CPC C11B 9/0073; C11B 9/0015; C11B 9/0019

(57) ABSTRACT

The present disclosure relates to fragrance compositions, particularly fragrance compositions having long-lasting trail performance. The present disclosure also relates to use of the fragrance compositions as well as perfumed consumer products containing the fragrance compositions.

### CROSS REFERENCE TO RELATED APPLICATIONS

The present application claims the benefit of priority of U.S. Provisional Application No. 63/354,535, filed Jun. 22, 2022, and of European Application No. 22187312.8, filed Jul. 27, 2022. The entire contents of these applications are explicitly incorporated herein by this reference.

### FIELD OF THE DISCLOSURE

The present disclosure relates to fragrance compositions, particularly fragrance compositions having long-lasting trail performance. The present disclosure also relates to use of the fragrance compositions as well as perfumed consumer products containing the fragrance compositions.

### BACKGROUND OF THE DISCLOSURE

Fragrance performance, particularly in the context of fine fragrances, can be divided into three main attributes: impact, long-lastingness, and projection, the latter comprised of ‘diffusion’ and trail characteristics. Projection (sometimes also referred to as ‘volume’ in the art) describes how well the fragrance can be perceived at a distance by other people.

The term ‘diffusion’ in the field of perfumery generally means how effectively the fragrance (or a fragrant ingredient) can be perceived in the ‘aura’, or, the immediate vicinity, of the fragrance source (the fragrance wearer). The aura, in this context, is defined by the space around the source of a fragrance, where diffusion from the vapour-liquid interface (i.e., liquid perfume on skin exposed to air) is a relevant fragrance transport mechanism. Such a definition is also present in the following publication: ‘A Novel Technology to Study the Emission of Fragrance from the Skin’, by Braja D. Mookherjee, Subha M. Patel, Robert W. Trenkle and Richard A. Wilson in Cosmetics & Toiletries (1998), 113(7), 53-56, 58-60.

Unlike aura, trail is associated with significant movement and air convection. This can be either the movement of a person wearing fragrance (walking) or the movement of the air around the person (wind or ventilation) or both (person walking in the street, exposed to natural wind currents). Movement induces a certain degree of air convection, transporting the fragrance away from the source (the person wearing fragrance) and allowing other people to smell the fragrance. Trail performance of fine fragrances is generally judged by the maximum distance from the source at which the fragrance remains at least perceptible, and preferably recognizable, to other people. It is important to note that, whenever significant gas-phase concentration gradients exist, such as with surrounding unscented air, the extent of fragrance dilution in the air is correlated strongly with distance from the fragrance source at which the evaluation is performed.

While fragrance trail is often referenced in both patent literature and scientific publications, there still exists a gap in the published art in terms of the link between performance at a distance and the fragrance ingredients or fragrance accords, including their olfactive dose-response characteristics, that elicit such performance. Selecting materials and methods to improve the trail performance of a fragrance is often necessary. In many fine fragrance markets, trail is a key consumer need and often a must-have. Thus, trail performance is becoming an integral part of the fragrance success.

Accordingly, there is an ongoing need for fragrance compositions that can elicit the desired long-lasting trail performance.

### SUMMARY OF THE DISCLOSURE

The following aspects of the present disclosure seek to address one or more of the problems described hereinabove.

In a first aspect, the present disclosure relates to a fragrance composition comprising at least 4%, typically at least 5%, more typically at least 6%, by weight relative to the total weight of the composition, of at least two group A ingredients, wherein the group A ingredients are described herein, and wherein each of the at least two group A ingredients have a volatility of less than 100 μg/L, typically from 100 μg/L to 10 μg/L or from 10 μg/L to 0.001 μg/L.

In a second aspect, the present disclosure relates to a perfumed consumer product comprising the fragrance composition described herein.

In a third aspect, the present disclosure relates to the use of the fragrance composition or the perfumed consumer product described herein to provide a long-lasting fragrance trail.

### BRIEF DESCRIPTION OF THE FIGURES

FIG. 1 shows the trail intensity of a comparative composition and a composition according to the present disclosure after a 30-minute drydown at 1 meter, 2 meters, and 3 meters.

FIG. 2 shows the trail intensity of a comparative composition and a composition according to the present disclosure after a 4-hour drydown at 1 meter, 2 meters, and 3 meters.

FIG. 3 shows the trail intensity of a comparative composition and a composition according to the present disclosure after a 30-minute drydown at 1 meter and 3 meters.

FIG. 4 shows the trail intensity of a comparative composition and a composition according to the present disclosure after a 4-hour drydown at 1 meter and 3 meters.

FIG. 5 shows the trail intensity of a floral chassis, an inventive product according to the present disclosure, and a commercial product after a 2-hour drydown at 1 meter and 3 meters.

### DETAILED DESCRIPTION

As used herein, the terms “a”, “an”, or “the” means “one or more” or “at least one” unless otherwise stated.

While compositions and methods are described in terms of “comprising,” “containing,” or “including” various components or steps, the compositions and methods can also “consist essentially of” or “consist of” the various components, substances and steps. As used herein the term “consisting essentially of” shall be construed to mean including the listed components, substances or steps and such additional components, substances or steps which do not materially affect the basic and novel properties of the composition or method. In some embodiments, a composition in accordance with embodiments of the present disclosure that “consists essentially of” the recited components or substances does not include any additional components or substances that alter the basic and novel properties of the composition.

Unless defined otherwise, all technical and scientific terms used herein have the same meaning as is commonly understood by one of skill in the art to which this specification pertains.

It should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Throughout the present disclosure, various publications may be incorporated by reference. Should the meaning of any language in such publications incorporated by reference conflict with the meaning of the language of the present disclosure, the meaning of the language of the present disclosure shall take precedence, unless otherwise indicated.

Throughout the present disclosure, various chemical names and structures may be recited. Unless otherwise stated, any stereoisomers, such as enantiomers, diastereomers, anomers, epimers, and the like; and geometric isomers, such as cis/trans or E/Z isomers, of the recited chemical name or structure are contemplated. As would be understood by those of ordinary skill in the art, stereoisomers may possess one stereocenter, giving rise to enantiomers, or more than one stereocenter, giving rise to diastereomers, each stereocenter having one of two different stereochemistries (i.e., R or S). Enantiomers may be characterized by their ability to rotate oncoming plane-polarized light to the right, designated as dextrorotatory, “(+)” or “D”, or to the left, designated as levorotatory, “(−)” or “L”. Enantiomers may exist as racemic mixtures or scalemic mixtures. Geometric isomers refer to isomers in which the spatial relationship of atoms around a double bond are different, typically designated E or Z according to conventional understanding in the chemical art. Geometric isomers may also exist as mixtures of E and Z isomers. All of the aforementioned isomeric variations of the chemical names or structures recited herein are included.

In the first aspect, the present disclosure relates to a fragrance composition comprising at least 4%, typically at least 5%, more typically at least 6%, by weight relative to the total weight of the composition, of at least two group A ingredients, wherein the group A ingredients are described herein, and wherein each of the at least two group A ingredients have a volatility of less than 100 μg/L, typically from 100 μg/L to 10 μg/L or from 10 μg/L to 0.001 μg/L.

It has been discovered that certain perfumery ingredients are capable of providing a high level of long-lasting trail performance when used in fragrance compositions. As used herein, perfumery ingredients providing a high level of long-lasting trail performance are categorized as group A ingredients. In addition to the ability to provide a high level of long-lasting trail performance, the group A ingredients may be further characterized by a volatility of less than 100 μg/L. While the group A ingredients may be characterized by volatility, it should be noted that volatility and the ability of an ingredient to provide a high level of long-lasting trail performance are not connected. Thus, the volatility of an ingredient does not give any indication of an ingredient's ability to provide a high level of long-lasting trail performance.

Group A ingredients having a volatility of less than 100 μg/L are selected from the group consisting of:
(3aRS,5aRS,8aRS,8bSR)-2,2,6,6,7,8,8-heptamethyldecahydro-2H-indeno[4,5-b]furan,
4-methoxybenzaldehyde,
(+−)-1-(5-propyl-1,3-benzodioxol-2-yl)ethenone,
3-(4-TERT-BUTYLPHENYL)PROPANAL,
3-BUTYLIDENE-1-BENZO[C]FURANONE,
7-methyl-2H-1,5-benzodioxepin-3(4H)-one,
5-ISOPROPYL-2-METHYLPHENOL,
7-ISOPROPYL-2H,4H-1,5-BENZODIOXEPIN-3-ONE,
4,8-cyclododecadien-1-one,
(E)-3-PHENYL-2-PROPENENITRILE,
3-methyl-5-phenyl-2-pentenenitrile,
(3S,3aR,5R,8S,8aS)-5-isopropenyl-3,8-dimethyloctahydro-3a(1H)-azulenol, isopropenyl-1,4-dimethyldecahydro-4-azulenol,
(1R,3R,6S,7S,8S)-2,2,6,8-tetramethyltricyclo[5.3.1.0˜3,8˜]undecan-3-ol,
(1R,3R,6S,7S,8S)-3-ethoxy-2,2,6,8-tetramethyltricyclo[5.3.1.0˜3,8˜]undecane,
(+−)-6-METHYL-7-OXA-1-THIA-4-AZASPIRO[4.4]NONANE,
4-METHYLPHENYL 3-METHYLBUTANOATE,
(2E)-1-(2,6,6-trimethyl-1,3-cyclohexadien-1-yl)-2-buten-1-one,
(+−)-(2E)-1-(2,6,6-trimethyl-2-cyclohexen-1-yl)-2-buten-1-one,
(2E)-2-dodecenal,
(+−)-4-methylene-2-phenyltetrahydro-2H-pyran,
(+−)-4-methyl-6-phenyl-3,6-dihydro-2H-pyran,
(+−)-4-methyl-2-phenyl-3,6-dihydro-2H-pyran,
(E)-3-METHYL-5-(2,2,3-TRIMETHYL-3-CYCLOPENTEN-1-YL)-4-PENTEN-2-OL,
ETHYL 2,3-EPOXY-3-PHENYLBUTANOATE,
2-ETHYL-3-HYDROXY-4 (4H)-PYRANONE,
2-methoxy-4-(2-propen-1-yl) phenol,
4-allyl-2-methoxyphenyl acetate,
2-methyl-4-[(1R)-2,2,3-trimethyl-3-cyclopenten-1-yl]-4-penten-1-ol,
(+−)-5-ethyl-4-hydroxy-2-methyl-3 (2H)-furanone,
(+−)-2-ethyl-4-hydroxy-5-methyl-3 (2H)-furanone,
4-METHYLPHENOXY)ACETALDEHYDE,
2-methoxy-4-[(1E)-1-propen-1-yl]phenol,
8-isopropylquinoline,
6-isopropylquinoline,
3,7-DIMETHYL-6-OCTEN-1-OL,
3,7-DIMETHYL-7-OCTEN-1-OL,
(+−)-2-methyl-3-[4-(2-methyl-2-propanyl)phenyl]propanal,
(+−)-2,4-dimethyl-4,4a,5,9b-tetrahydroindeno[1,2-d][1,3]dioxine,
3,12-TRIDECADIENENITRILE,
methyl 2-aminobenzoate,
2,6-DIMETHOXYPHENOL,
3,6-dimethylhexahydro-1-benzofuran-2(3H)-one,
1-(5,5-dimethyl-1-cyclohexen-1-yl)-4-penten-1-one,
(+−)-1-(5-ethyl-5-methyl-1-cyclohexen-1-yl)-4-penten-1-one,
1-(3-METHYL-1-BENZOFURAN-2-YL)ETHANONE,
5-NONANOLIDE,
(+−)-4-NONANOLIDE,
PERHYDRO-2-CHROMENONE,
1-(2-AMINOPHENYL)-1-ETHANONE,
(−)-(1R,3R,6S,7S,8S)-2,2,6,8-tetramethyltricyclo[5.3.1.0˜3,8˜]undecan-3-ol,
(E)-2-ETHOXY-5-(1-PROPENYL)PHENOL,
3-METHYLINDOLE,
1-oxaspiro[4.5]decan-2-one,
(4Z)-4-dodecenal,
1,5,9-TRIMETHYL-4,8-DECADIENYL ACETATE,
3-METHYL-4-OCTANOLIDE,
3-PROPYLPHENOL,
2-METHOXYNAPHTHALENE,
INDOLE,
5-ETHYL-3-HYDROXY-4-METHYL-2 (5H)-FURANONE,
(+−)-2-[4-(2-hydroxy-2-methylpropyl)phenyl]propanal,
3-(3-(2-hydroxy-2-methylpropyl)phenyl)propanal,
3-[4-(2-hydroxy-2-methylpropyl)phenyl]propanal,
4-(4-methoxyphenyl)-2-butanone,
2-(3-phenylpropyl)pyridine,
(−)-(2E)-2-ethyl-4-[(1R)-2,2,3-trimethyl-3-cyclopenten-1-yl]-2-buten-1-ol,
(+−)-6-pentyltetrahydro-2H-pyran-2-one,
3-(3,3-dimethyl-2,3-dihydro-1H-inden-5-yl)propanal
3-(1,1-dimethyl-2,3-dihydro-1H-inden-4-yl)propanal
3-(1,1-dimethyl-2,3-dihydro-1H-inden-5-yl)propanal,
(E)-2-METHOXY-4-(1-PROPENYL)PHENYL ACETATE,
(1'S,3′R)-{1-METHYL-2-[(1′,2′,2′-TRIMETHYLBICYCLO[3.1.0]HEX-3′-YL)METHYL]CYCLOPROPYL}METHANOL,
(+−)-(2,5-dimethyl-2,3-dihydro-1H-inden-2-yl)methanol,
(+−)-3-(4-hydroxy-4-methylpentyl)-3-cyclohexene-1-carbaldehyde,
(+−)-4-(4-hydroxy-4-methylpentyl)-3-cyclohexene-1-carbaldehyde,
METHYL 2,4-DIHYDROXY-3,6-DIMETHYLBENZOATE,
4-(4-hydroxyphenyl)-2-butanone,
(+)-(1R,7R)-10,10-DIMETHYL-TRICYCLO[7.1.1.0(2,7)]UNDEC-2-EN-4-ONE,
(+−)-5-heptyldihydro-2(3H)-furanone,
4-hydroxy-3-methoxybenzaldehyde,
(−)-(3aR,5aS,9aS,9bR)-3a,6,6,9a-tetramethyldodecahydronaphtho[2,1-b]furan,
(−)-(1R,3S,7R,8R,10S,13R)-5,5,7,9,9,13-HEXAMETHYL-4,6-DIOXATETRACYCLO[6.5.1.0(1,10).0(3,7)]TETRADECANE,
3-ethoxy-4-hydroxybenzaldehyde, and any combination thereof.

The group A ingredients described herein may have a volatility from 100 μg/L to 10 μg/L or from 10 μg/L to 0.001 μg/L.

The group A ingredients described herein having a volatility from 100 μg/L to 10 μg/L are those selected from the group consisting of:
(3aRS,5aRS,8aRS,8bSR)-2,2,6,6,7,8,8-heptamethyldecahydro-2H-indeno[4,5-b]furan,
4-methoxybenzaldehyde,
(+−)-1-(5-propyl-1,3-benzodioxol-2-yl)ethenone,
3-(4-TERT-BUTYLPHENYL)PROPANAL,
3-BUTYLIDENE-1-BENZO[C]FURANONE,
7-methyl-2H-1,5-benzodioxepin-3(4H)-one,
5-ISOPROPYL-2-METHYLPHENOL,
7-ISOPROPYL-2H,4H-1,5-BENZODIOXEPIN-3-ONE,
4,8-cyclododecadien-1-one,
(E)-3-PHENYL-2-PROPENENITRILE,
3-methyl-5-phenyl-2-pentenenitrile,
(3S,3aR,5R,8S,8aS)-5-isopropenyl-3,8-dimethyloctahydro-3a(1H)-azulenol, isopropenyl-1,4-dimethyldecahydro-4-azulenol,
(1R,3R,6S,7S,8S)-2,2,6,8-tetramethyltricyclo[5.3.1.0˜3,8˜]undecan-3-ol,
(1R,3R,6S,7S,8S)-3-ethoxy-2,2,6,8-tetramethyltricyclo[5.3.1.0˜3,8˜]undecane,
(+−)-6-METHYL-7-OXA-1-THIA-4-AZASPIRO[4.4]NONANE,
4-METHYLPHENYL 3-METHYLBUTANOATE,
(2E)-1-(2,6,6-trimethyl-1,3-cyclohexadien-1-yl)-2-buten-1-one,
(+−)-(2E)-1-(2,6,6-trimethyl-2-cyclohexen-1-yl)-2-buten-1-one,
(2E)-2-dodecenal,
(+−)-4-methylene-2-phenyltetrahydro-2H-pyran,
(+−)-4-methyl-6-phenyl-3,6-dihydro-2H-pyran,
(+−)-4-methyl-2-phenyl-3,6-dihydro-2H-pyran,
(E)-3-METHYL-5-(2,2,3-TRIMETHYL-3-CYCLOPENTEN-1-YL)-4-PENTEN-2-OL,
ETHYL 2,3-EPOXY-3-PHENYLBUTANOATE,
2-ETHYL-3-HYDROXY-4 (4H)-PYRANONE,
2-methoxy-4-(2-propen-1-yl) phenol,
4-allyl-2-methoxyphenyl acetate,
2-methyl-4-[(1R)-2,2,3-trimethyl-3-cyclopenten-1-yl]-4-penten-1-ol,
(+−)-5-ethyl-4-hydroxy-2-methyl-3 (2H)-furanone,
(+−)-2-ethyl-4-hydroxy-5-methyl-3 (2H)-furanone,
4-METHYLPHENOXY)ACETALDEHYDE,
2-methoxy-4-[(1E)-1-propen-1-yl]phenol,
8-isopropylquinoline,
6-isopropylquinoline,
3,7-DIMETHYL-6-OCTEN-1-OL,
3,7-DIMETHYL-7-OCTEN-1-OL,
(+−)-2-methyl-3-[4-(2-methyl-2-propanyl)phenyl]propanal,
(+−)-2,4-dimethyl-4,4a,5,9b-tetrahydroindeno[1,2-d][1,3]dioxine,
3,12-TRIDECADIENENITRILE,
methyl 2-aminobenzoate,
2,6-DIMETHOXYPHENOL,
3,6-dimethylhexahydro-1-benzofuran-2(3H)-one,
1-(5,5-dimethyl-1-cyclohexen-1-yl)-4-penten-1-one,
(+−)-1-(5-ethyl-5-methyl-1-cyclohexen-1-yl)-4-penten-1-one,
1-(3-METHYL-1-BENZOFURAN-2-YL)ETHANONE,
5-NONANOLIDE,
(+−)-4-NONANOLIDE,
PERHYDRO-2-CHROMENONE,
1-(2-AMINOPHENYL)-1-ETHANONE,
(−)-(1R,3R,6S,7S,8S)-2,2,6,8-tetramethyltricyclo[5.3.1.0˜3,8˜]undecan-3-ol,
(E)-2-ETHOXY-5-(1-PROPENYL)PHENOL,
3-METHYLINDOLE,
1-oxaspiro[4.5]decan-2-one,
(4Z)-4-dodecenal,
1,5,9-TRIMETHYL-4,8-DECADIENYL ACETATE,
3-METHYL-4-OCTANOLIDE,
3-PROPYLPHENOL,
2-METHOXYNAPHTHALENE,
INDOLE,
5-ETHYL-3-HYDROXY-4-METHYL-2(5H)-FURANONE, and combinations thereof.

The group A ingredients described herein having a volatility from 10 μg/L to 0.001 μg/L are those selected from the group consisting of:
(+−)-2-[4-(2-hydroxy-2-methylpropyl)phenyl]propanal,
3-(3-(2-hydroxy-2-methylpropyl)phenyl)propanal,
3-[4-(2-hydroxy-2-methylpropyl)phenyl]propanal,
4-(4-methoxyphenyl)-2-butanone,
2-(3-phenylpropyl)pyridine,
(−)-(2E)-2-ethyl-4-[(1R)-2,2,3-trimethyl-3-cyclopenten-1-yl]-2-buten-1-ol,
(+−)-6-pentyltetrahydro-2H-pyran-2-one,
3-(3,3-dimethyl-2,3-dihydro-1H-inden-5-yl)propanal
3-(1,1-dimethyl-2,3-dihydro-1H-inden-4-yl)propanal
3-(1,1-dimethyl-2,3-dihydro-1H-inden-5-yl)propanal,
(E)-2-METHOXY-4-(1-PROPENYL)PHENYL ACETATE,
(1'S,3′R)-{1-METHYL-2-[(1′,2′,2′-TRIMETHYLBICYCLO[3.1.0]HEX-3′-YL)METHYL]CYCLOPROPYL}METHANOL,
(+−)-(2,5-dimethyl-2,3-dihydro-1H-inden-2-yl)methanol;
(+−)-3-(4-hydroxy-4-methylpentyl)-3-cyclohexene-1-carbaldehyde,
(+−)-4-(4-hydroxy-4-methylpentyl)-3-cyclohexene-1-carbaldehyde,
METHYL 2,4-DIHYDROXY-3,6-DIMETHYLBENZOATE,
4-(4-hydroxyphenyl)-2-butanone,
(+)-(1R,7R)-10,10-DIMETHYL-TRICYCLO[7.1.1.0(2,7)]UNDEC-2-EN-4-ONE,
(+−)-5-heptyldihydro-2(3H)-furanone,
4-hydroxy-3-methoxybenzaldehyde,
(−)-(3aR,5aS,9aS,9bR)-3a,6,6,9a-tetramethyldodecahydronaphtho[2,1-b]furan,
(−)-(1R,3S,7R,8R,10S,13R)-5,5,7,9,9,13-HEXAMETHYL-4,6-DIOXATETRACYCLO[6.5.1.0(1,10).0(3,7)]TETRADECANE, 3-ethoxy-4-hydroxybenzaldehyde, and any combination thereof.

The fragrance composition comprises at least 4%, typically at least 5%, more typically at least 6%, by weight relative to the total weight of the composition, of at least two group A ingredients. In an embodiment, the at least two group A ingredients are present in an amount of from 4% to 95%, typically from 5% to 80%, more typically from 6% to 70%, by weight relative to the total weight of the composition.

The fragrance composition according to the present disclosure may further comprise at least 1%, typically at least 3%, more typically at least 4%, by weight relative to the total weight of the composition, of at least two group B ingredients, wherein each of the at least two group B ingredients have a volatility of less than 100 μg/L, typically from 100 μg/L to 10 μg/L or from 10 μg/L to 0.001 μg/L.

The group B ingredients described herein are perfumery ingredients that can provide a desirable level of long-lasting trail performance when used in a fragrance composition. In addition to the ability to provide a desirable level of long-lasting trail performance, the group B ingredients may be further characterized by a volatility of less than 100 μg/L. As with the group A ingredients, while the group B ingredients may be characterized by volatility, it should be noted that volatility and the ability of the ingredient to provide a desirable level of long-lasting trail performance are not connected. Thus, the volatility of a group B ingredient does not give any indication of the ingredient's ability to provide a desirable level of long-lasting trail performance.

Group B ingredients having a volatility of less than 100 μg/L are selected from the group consisting of:
1-(4-METHOXYPHENYL)-1-ETHANONE,
(+−)-8alpha,8A-EPOXY-PERHYDRO-2beta,5,5-TRIMETHYL-2-TRANS-NAPHTHALENOL,
(+−)-4A,8A-EPOXY-PERHYDRO-2,5,5-TRIMETHYL-2-NAPHTHALENOL,
(+−)-4-(C-2,3-EPOXY-2,6,6-TRIMETHYL-R-1-CYCLOHEXYL)-2-BUTANONE,
4-METHOXYBENZYL ACETATE,
4-METHOXY-1-BENZENEMETHANOL,
BENZYL (E)-2-METHYL-2-BUTENOATE,
(+−)-1,1,2,3,3-pentamethyl-1,2,3,5,6,7-hexahydro-4H-inden-4-one,
cedr-8-ene,
1-(cedr-8-en-9-yl)ethenone,
(2E)-3-phenyl-2-propen-1-ol,
(E)-3-PHENYL-2-PROPENAL,
(E)-3-PHENYL-2-PROPENYL ACETATE,
3-methyl-2-[(2Z)-2-penten-1-yl]-2-cyclopenten-1-one,
3-hydroxy-2-methyl-4H-pyran-4-one,
ALLYL (CYCLOHEXYLOXY)ACETATE,
2-methoxy-4-propylphenol,
4-methyl-2-phenyltetrahydro-2H-pyran,
(2E)-1-(2,6,6-trimethyl-1-cyclohexen-1-yl)-2-buten-1-one,
ETHYL (E)-3-PHENYL-2-PROPENOATE,
3-(4-ETHYLPHENYL)-2,2-DIMETHYLPROPANAL,
3-(2-ETHYLPHENYL)-2,2-DIMETHYLPROPANAL,
(+−)-3-(3-ISOPROPYL-1-PHENYL) BUTANAL,
(+−)-(E)-3-METHYL-4-(2,6,6-TRIMETHYL-2-CYCLOHEXEN-1-YL)-3-BUTEN-2-ONE,
(E)-1-(2,6,6-TRIMETHYL-2-CYCLOHEXEN-1-YL)-1-PENTEN-3-ONE,
(+−)-(E)-1-(2,2-DIMETHYL-6-METHYLENE-1-CYCLOHEXYL)-1-PENTEN-3-ONE,
(E)-1-(2,6,6-TRIMETHYL-1-CYCLOHEXEN-1-YL)-1-PENTEN-3-ONE,
(E)-3,7-DIMETHYL-2,6-OCTADIEN-1-OL,
benzo[d][1,3]dioxole-5-carbaldehyde,
(+−)-(3E)-3-methyl-4-(2,6,6-trimethyl-2-cyclohexen-1-yl)-3-buten-2-one,
(+−)-(1E)-1-(2,6,6-trimethyl-2-cyclohexen-1-yl)-1-penten-3-one,
(+−)-(E)-TRANS-alpha-IRONE,
(+−)-(E)-CIS-alpha-IRONE,
(+−)-(E)-beta-IRONE,
(+−)-(3E)-3-methyl-4-(2,6,6-trimethyl-2-cyclohexen-1-yl)-3-buten-2-one,
(+−)-(1E)-1-(2,6,6-trimethyl-2-cyclohexen-1-yl)-1-penten-3-one,
3,7-dimethyl-2,6-nonadienenitrile,
3,7-dimethyl-3,6-nonadienenitrile,
10-METHYL-alpha-IONONE,
10-METHYL-beta-IONONE,
10-METHYL-GAMMA-IONONE,
(1E)-1-(2,6,6-trimethyl-1-cyclohexen-1-yl)-1-penten-3-one,
(+)-3,3-dimethyl-5-[(1S)-2,2,3-trimethyl-3-cyclopenten-1-yl]-4-penten-2-ol,
(+−)-6-propyltetrahydro-2H-pyran-2-one,
(1RS,4aSR,8RS,8aSR)-2,2,6,8-tetramethyl-1,2,3,4,4a,5,8,8a-octahydro-1-naphthalenol,
(3Z)-3-hexen-1-yl hexanoate,
2,4,6,8-TETRAMETHYLNONYL ACETATE,
(−)-3,3-dimethyl-5-[(1R)-2,2,3-trimethyl-3-cyclopenten-1-yl]-4-penten-2-ol,
(2RS,4aRS,8aSR)-5,5,8a-trimethyldecahydro-2-naphthalenyl acetate,
(2S,5R)-5-methyl-2-(2-propanyl)cyclohexanone oxime,
2,6,10,10-tetramethyl-1-oxaspiro[4.5]decan-6-ol,
(4Z)-4-DODECENENITRILE,
DODECANENITRILE,
Thymol,
TRICYCLO[5.2.1.0(2,6)]DEC-3-EN-8-YL PROPANOATE,
TRICYCLO[5.2.1.0(2,6)]DEC-4-EN-8-YL PROPANOATE,
2-TERT-BUTYL-1,4-DIMETHOXYBENZENE,
(+−)-(3E)-4-(2,6,6-trimethyl-2-cyclohexen-1-yl)-3-buten-2-one,
(3E)-4-(2,6,6-trimethyl-1-cyclohexen-1-yl)-3-buten-2-one,
4-methyl-6-phenyl-2-hexanol,
9-hydroxy-5,9-dimethyl-4-decenal;
7-PROPYL-2H,4H-1,5-BENZODIOXEPIN-3-ONE,
ALLYL PHENOXYACETATE,
(3aRS,5aSR,9aSR,9bRS)-3a,6,6,9a-tetramethyldodecahydronaphtho[2,1-b]furan,
(3aRS,5aSR,9aSR,9bSR)-3a,6,6,9a-tetramethyldodecahydronaphtho[2,1-b]furan,
(3aRS,5aSR,9aSR,9bRS)-3a,6,6,9a-tetramethyldodecahydronaphtho[2,1-b]furan,
(3aRS,5aSR,9aSR,9bSR)-3a,6,6,9a-tetramethyldodecahydronaphtho[2,1-b]furan,
(+−)-2-ETHYL-4-(2,2,3-TRIMETHYL-3-CYCLOPENTEN-1-YL)-2-BUTEN-1-OL,
(E)-3-PHENYL-2-PROPENYL PROPANOATE,
methyl [3-oxo-2-pentylcyclopentyl]acetate,
2-CHROMENONE,
(+−)-4-DECANOLIDE,
(+−)-5-octyldihydro-2(3H)-furanone,
(+−)-6-heptyltetrahydro-2H-pyran-2-one,
(+−)-3-(1,3-BENZODIOXOL-5-YL)-2-METHYLPROPANAL,
3-(3,3-dimethyl-2,3-dihydro-1H-inden-5-yl)propanal,
3-(1,1-dimethyl-2,3-dihydro-1H-inden-5-yl)propanal,
4-formyl-2-methoxyphenyl 2-methylpropanoate,
(+−)-8-sec-butylquinoline,
(+−)-6-sec-butylquinoline,
(+−)-6-[(2Z)-2-penten-1-yl]tetrahydro-2H-pyran-2-one,
2-ethoxy-4-(methoxymethyl) phenol,
1-(2-naphthyl)ethenone,
(4E)-4-methyl-5-(4-methylphenyl)-4-pentenal,
2-{2-[(1R)-4-methyl-3-cyclohexen-1-yl]propyl}cyclopentanone,
(+)-(4R,4aS,6R)-4,4a-dimethyl-6-(1-propen-2-yl)-4,4a,5,6,7,8-hexahydro-2(3H)-naphthalenone,
1-[2,2,6-trimethylcyclohexyl]-3-hexanol,
2-tridecenenitrile,
3-tridecenenitrile,
4-METHYLPHENYL PHENYLACETATE,
(+−)-3-methyl-5-phenyl-1-pentanol,
(+−)-2,3,3-TRIMETHYL-1-INDANONE,
2-(4-METHYL-1,3-THIAZOL-5-YL)-1-ETHANOL,
7-(2-methyl-2-propanyl)-2H-1,5-benzodioxepin-3(4H)-one,
(E)-2-TRIDECENAL,
6-hexyltetrahydro-2H-pyran-2-one,
2-methoxy-4-methylphenyl methyl carbonate,
(+)-(1S,2S,3S,5R)-2,6,6-trimethylspiro[bicyclo[3.1.1]heptane-3,1′-cyclohexane]-2′-en-4′-one,
(1S,4S,9S,10R,13R)-5,5,9,13-tetramethyl-14,16-dioxatetracyclo[11.2.1.0˜1,10˜0.0˜4,9˜]hexadecane,
(1R,4S,9S,10R,13S)-5,5,9,13-tetramethyl-14,16-dioxatetracyclo[11.2.1.0˜1,10˜0.0˜4,9˜]hexadecane, and any combination thereof;

The group B ingredients described herein may have a volatility from 100 μg/L to 10 μg/L or from 10 μg/L to 0.001 μg/L.

The group B ingredients described herein having a volatility from 100 μg/L to 10 μg/L are those selected from the group consisting of:
1-(4-METHOXYPHENYL)-1-ETHANONE,
(+−)-8alpha,8A-EPOXY-PERHYDRO-2beta,5,5-TRIMETHYL-2-TRANS-NAPHTHALENOL,
(+−)-4A,8A-EPOXY-PERHYDRO-2,5,5-TRIMETHYL-2-NAPHTHALENOL,
(+−)-4-(C-2,3-EPOXY-2,6,6-TRIMETHYL-R-1-CYCLOHEXYL)-2-BUTANONE,
4-METHOXYBENZYL ACETATE,
4-METHOXY-1-BENZENEMETHANOL,
BENZYL (E)-2-METHYL-2-BUTENOATE,
(+−)-1,1,2,3,3-pentamethyl-1,2,3,5,6,7-hexahydro-4H-inden-4-one,
cedr-8-ene,
1-(cedr-8-en-9-yl)ethenone,
(2E)-3-phenyl-2-propen-1-ol,
(E)-3-PHENYL-2-PROPENAL,
(E)-3-PHENYL-2-PROPENYL ACETATE,
3-methyl-2-[(2Z)-2-penten-1-yl]-2-cyclopenten-1-one,
3-hydroxy-2-methyl-4H-pyran-4-one,
ALLYL (CYCLOHEXYLOXY)ACETATE,
2-methoxy-4-propylphenol,
4-methyl-2-phenyltetrahydro-2H-pyran,
(2E)-1-(2,6,6-trimethyl-1-cyclohexen-1-yl)-2-buten-1-one,
ETHYL (E)-3-PHENYL-2-PROPENOATE,
3-(4-ETHYLPHENYL)-2,2-DIMETHYLPROPANAL,
3-(2-ETHYLPHENYL)-2,2-DIMETHYLPROPANAL,
(+−)-3-(3-ISOPROPYL-1-PHENYL) BUTANAL,
(+−)-(E)-3-METHYL-4-(2,6,6-TRIMETHYL-2-CYCLOHEXEN-1-YL)-3-BUTEN-2-ONE,
(E)-1-(2,6,6-TRIMETHYL-2-CYCLOHEXEN-1-YL)-1-PENTEN-3-ONE,
(+−)-(E)-1-(2,2-DIMETHYL-6-METHYLENE-1-CYCLOHEXYL)-1-PENTEN-3-ONE,
(E)-1-(2,6,6-TRIMETHYL-1-CYCLOHEXEN-1-YL)-1-PENTEN-3-ONE,
(E)-3,7-DIMETHYL-2,6-OCTADIEN-1-OL,
benzo[d][1,3]dioxole-5-carbaldehyde,
(+−)-(3E)-3-methyl-4-(2,6,6-trimethyl-2-cyclohexen-1-yl)-3-buten-2-one,
(+−)-(1E)-1-(2,6,6-trimethyl-2-cyclohexen-1-yl)-1-penten-3-one,
(+−)-(E)-TRANS-alpha-IRONE,
(+−)-(E)-CIS-alpha-IRONE,
(+−)-(E)-beta-IRONE,
(+−)-(3E)-3-methyl-4-(2,6,6-trimethyl-2-cyclohexen-1-yl)-3-buten-2-one,
(+−)-(1E)-1-(2,6,6-trimethyl-2-cyclohexen-1-yl)-1-penten-3-one,
3,7-dimethyl-2,6-nonadienenitrile,
3,7-dimethyl-3,6-nonadienenitrile,
10-METHYL-alpha-IONONE,
10-METHYL-beta-IONONE,
10-METHYL-GAMMA-IONONE,
(1E)-1-(2,6,6-trimethyl-1-cyclohexen-1-yl)-1-penten-3-one,
(+)-3,3-dimethyl-5-[(1S)-2,2,3-trimethyl-3-cyclopenten-1-yl]-4-penten-2-ol,
(+−)-6-propyltetrahydro-2H-pyran-2-one,
(1RS,4aSR,8RS,8aSR)-2,2,6,8-tetramethyl-1,2,3,4,4a,5,8,8a-octahydro-1-naphthalenol,
(3Z)-3-hexen-1-yl hexanoate,
2,4,6,8-TETRAMETHYLNONYL ACETATE,
(−)-3,3-dimethyl-5-[(1R)-2,2,3-trimethyl-3-cyclopenten-1-yl]-4-penten-2-ol,
(2RS,4aRS,8aSR)-5,5,8a-trimethyldecahydro-2-naphthalenyl acetate,
(2S,5R)-5-methyl-2-(2-propanyl)cyclohexanone oxime,
2,6,10,10-tetramethyl-1-oxaspiro[4.5]decan-6-ol,
(4Z)-4-DODECENENITRILE,
DODECANENITRILE,
Thymol,
TRICYCLO[5.2.1.0(2,6)]DEC-3-EN-8-YL PROPANOATE,
TRICYCLO[5.2.1.0(2,6)]DEC-4-EN-8-YL PROPANOATE,
2-TERT-BUTYL-1,4-DIMETHOXYBENZENE,
(+−)-(3E)-4-(2,6,6-trimethyl-2-cyclohexen-1-yl)-3-buten-2-one,
(3E)-4-(2,6,6-trimethyl-1-cyclohexen-1-yl)-3-buten-2-one,
4-methyl-6-phenyl-2-hexanol,
9-hydroxy-5,9-dimethyl-4-decenal,
and any combination thereof.

The group B ingredients described herein having a volatility from 10 μg/L to 0.001 μg/L are those selected from the group consisting of:
7-PROPYL-2H,4H-1,5-BENZODIOXEPIN-3-ONE,
ALLYL PHENOXYACETATE,
(3aRS,5aSR,9aSR,9bRS)-3a,6,6,9a-tetramethyldodecahydronaphtho[2,1-b]furan,
(3aRS,5aSR,9aSR,9bSR)-3a,6,6,9a-tetramethyldodecahydronaphtho[2,1-b]furan,
(3aRS,5aSR,9aSR,9bRS)-3a,6,6,9a-tetramethyldodecahydronaphtho[2,1-b]furan,
(3aRS,5aSR,9aSR,9bSR)-3a,6,6,9a-tetramethyldodecahydronaphtho[2,1-b]furan,
(+−)-2-ETHYL-4-(2,2,3-TRIMETHYL-3-CYCLOPENTEN-1-YL)-2-BUTEN-1-OL,
(E)-3-PHENYL-2-PROPENYL PROPANOATE,
methyl [3-oxo-2-pentylcyclopentyl]acetate,
2-CHROMENONE,
(+−)-4-DECANOLIDE,
(+−)-5-octyldihydro-2(3H)-furanone,
(+−)-6-heptyltetrahydro-2H-pyran-2-one,
(+−)-3-(1,3-BENZODIOXOL-5-YL)-2-METHYLPROPANAL,
3-(3,3-dimethyl-2,3-dihydro-1H-inden-5-yl)propanal,
3-(1,1-dimethyl-2,3-dihydro-1H-inden-5-yl)propanal,
4-formyl-2-methoxyphenyl 2-methylpropanoate,
(+−)-8-sec-butylquinoline,
(+−)-6-sec-butylquinoline,
(+−)-6-[(2Z)-2-penten-1-yl]tetrahydro-2H-pyran-2-one,
2-ethoxy-4-(methoxymethyl) phenol,
1-(2-naphthyl)ethenone,
(4E)-4-methyl-5-(4-methylphenyl)-4-pentenal,
2-{2-[(1R)-4-methyl-3-cyclohexen-1-yl]propyl}cyclopentanone,
(+)-(4R,4aS,6R)-4,4a-dimethyl-6-(1-propen-2-yl)-4,4a,5,6,7,8-hexahydro-2(3H)-naphthalenone,
1-[2,2,6-trimethylcyclohexyl]-3-hexanol,
2-tridecenenitrile,
3-tridecenenitrile,
4-METHYLPHENYL PHENYLACETATE,
(+−)-3-methyl-5-phenyl-1-pentanol,
(+−)-2,3,3-TRIMETHYL-1-INDANONE,
2-(4-METHYL-1,3-THIAZOL-5-YL)-1-ETHANOL,
7-(2-methyl-2-propanyl)-2H-1,5-benzodioxepin-3(4H)-one,
(E)-2-TRIDECENAL,
6-hexyltetrahydro-2H-pyran-2-one,
2-methoxy-4-methylphenyl methyl carbonate,
(+)-(1S,2S,3S,5R)-2,6,6-trimethylspiro[bicyclo[3.1.1]heptane-3,1′-cyclohexane]-2′-en-4′-one,
   (1S,4S,9S,10R,13R)-5,5,9,13-tetramethyl-14,16-dioxatetracyclo[11.2.1.0˜1,10˜0.0˜4,9˜]hexadecane,
   (1R,4S,9S,10R,13S)-5,5,9,13-tetramethyl-14,16-dioxatetracyclo[11.2.1.0˜1,10˜0.0˜4,9˜]hexadecane, and any combination thereof.

In an embodiment, the at least two group B ingredients are present in an amount of at least 1%, typically at least 3%, more typically at least 4%, by weight relative to the total weight of the composition. In another embodiment, the at least two group B ingredients are present in an amount of from 1% to 95%, typically from 3% to 80%, more typically from 4% to 70%, by weight relative to the total weight of the composition.

In an embodiment, at least one of the at least two group A ingredients and/or at least one of the at least two group B ingredients have a volatility of from 10 μg/L to 0.001 μg/L. In another embodiment, at least one of the at least two group A ingredients and at least one of the at least two group B ingredients have a volatility of from 10 μg/L to 0.001 μg/L. In yet another embodiment, at least one of the at least two group A ingredients or at least one of the at least two group B ingredients have a volatility of from 10 μg/L to 0.001 μg/L.

In an embodiment, at least one of the two group A ingredients and/or at least one of the at least two group B ingredients have a volatility of from 100 μg/L to 10 μg/L. In another embodiment, at least one of the two group A ingredients and at least one of the at least two group B ingredients have a volatility of from 100 μg/L to 10 μg/L. In yet another embodiment, at least one of the two group A ingredients or at least one of the at least two group B ingredients have a volatility of from 100 μg/L to 10 μg/L.

Certain group A ingredients are particularly useful when used in the fragrance composition described herein. In an embodiment, at least one of the at least two group A ingredients is selected from the group consisting of:
(+−)-2-[4-(2-hydroxy-2-methylpropyl)phenyl]propanal,
3-(3-(2-hydroxy-2-methylpropyl)phenyl)propanal,
3-[4-(2-hydroxy-2-methylpropyl)phenyl]propanal,
(+−)-1-(5-propyl-1,3-benzodioxol-2-yl)ethenone,
(3S,3aR,5R,8S,8aS)-5-isopropenyl-3,8-dimethyloctahydro-3a(1H)-azulenol,
7-isopropenyl-1,4-dimethyldecahydro-4-azulenol,
(1R,3R,6S,7S,8S)-2,2,6,8-tetramethyltricyclo[5.3.1.0˜3,8˜]undecan-3-ol,
(1R,3R,6S,7S,8S)-3-ethoxy-2,2,6,8-tetramethyltricyclo[5.3.1.0˜3,8˜]undecane,
(+−)-6-METHYL-7-OXA-1-THIA-4-AZASPIRO[4.4]NONANE,
(2E)-2-dodecenal,
(+−)-4-methylene-2-phenyltetrahydro-2H-pyran,
(+−)-4-methyl-6-phenyl-3,6-dihydro-2H-pyran,
(+−)-4-methyl-2-phenyl-3,6-dihydro-2H-pyran,
3,6-dimethylhexahydro-1-benzofuran-2(3H)-one,
(+−)-1-(5-ethyl-5-methyl-1-cyclohexen-1-yl)-4-penten-1-one,
(−)-(1R,3R,6S,7S,8S)-2,2,6,8-tetramethyltricyclo[5.3.1.0˜3,8˜]undecan-3-ol,
1-oxaspiro[4.5]decan-2-one,
(4Z)-4-dodecenal,
3-PROPYLPHENOL, and any combination thereof.

Certain group B ingredients are also particularly useful when used in the fragrance composition described herein. In an embodiment, at least one of the at least two group B ingredients is selected from the group consisting of:
7-PROPYL-2H,4H-1,5-BENZODIOXEPIN-3-ONE,
(4E)-4-methyl-5-(4-methylphenyl)-4-pentenal,
7-(2-methyl-2-propanyl)-2H-1,5-benzodioxepin-3(4H)-one,
2-methoxy-4-methylphenyl methyl carbonate,
(+)-(1S,2S,3S,5R)-2,6,6-trimethylspiro[bicyclo[3.1.1]heptane-3,1′-cyclohexane]-2′-en-4′-one,
(1S,4S,9S,10R,13R)-5,5,9,13-tetramethyl-14,16-dioxatetracyclo[11.2.1.0˜1,10˜0.0˜4,9˜]hexadecane,
(1R,4S,9S,10R,13S)-5,5,9,13-tetramethyl-14,16-dioxatetracyclo[11.2.1.0˜1,10˜0.0˜4,9˜]hexadecane,
(+)-3,3-dimethyl-5-[(1S)-2,2,3-trimethyl-3-cyclopenten-1-yl]-4-penten-2-ol,
(1RS,4aSR,8RS,8aSR)-2,2,6,8-tetramethyl-1,2,3,4,4a,5,8,8a-octahydro-1-naphthalenol,
(2RS,4aRS,8aSR)-5,5,8a-trimethyldecahydro-2-naphthalenyl acetate,
(2S,5R)-5-methyl-2-(2-propanyl)cyclohexanone oxime,
2,6,10,10-tetramethyl-1-oxaspiro[4.5]decan-6-ol,
4-methyl-6-phenyl-2-hexanol, and any combination thereof.

The fragrance compositions may further comprise at least 0.1%, typically at least 2%, more typically at least 4%, by weight relative to the total weight of the composition, of at least two ingredients selected from group A′ ingredients, group B′ ingredients, or a combination thereof, each of the at least two ingredients having a volatility in the range of from 1000 μg/L to 100 μg/L, wherein the group A′ ingredients are selected from the group consisting of:
1-(PYRAZINYL)-1-ETHANONE,
(+−)-2-methylundecanal,
10-undecenal,
9-undecenal,
ALLYL (3-METHYLBUTOXY)ACETATE,
(+−)-ALLYL (2-METHYLBUTOXY)ACETATE,
(−)-(5R)-5-isopropenyl-2-methyl-2-cyclohexen-1-one,
(+−)-3-mercaptohexyl acetate,
(Z)-4-DECENAL,
(2E)-1-[(1RS,2SR)-2,6,6-trimethyl-3-cyclohexen-1-yl]-2-buten-1-one,
4-ETHYL-2-METHOXYPHENOL,
ETHYL 4,6,6-TRIMETHYL-1,3-CYCLOHEXADIENE-1-CARBOXYLATE,
(+−)-4-HYDROXY-2,5-DIMETHYL-3 (2H)-FURANONE,
(3E,5Z)-1,3,5-UNDECATRIENE,
(+−)-PERHYDRO-4alpha, 8Abeta-DIMETHYL-4A-NAPHTHALENOL,
2-METHOXYPHENOL,
8-ISOPROPYL-6-METHYL-BICYCLO[2.2.2]OCT-5-ENE-2-CARBALDEHYDE,
2-HEXYL-2-CYCLOPENTEN-1-ONE,
2-METHOXY-3-(4-METHYLPENTYL)PYRAZINE,
METHYL 2-OCTYNOATE,
1-(4-methylphenyl)ethenone,
(+−)-2-methyldecanal,
(2E,6Z)-1,1-DIETHOXY-2,6-NONADIENE,
(Z)-2-NONENAL,
2-HYDROXY-3-METHYL-2-CYCLOPENTEN-1-ONE,
GAMMA OCTALACTONE,
2,6,6-TRIMETHYL-2-CYCLOHEXENE-1,4-DIONE,
4-METHYLPHENOL,
4-METHYLPHENYL ACETATE,
4-METHYLPHENYL ISOBUTYRATE,
(+−)-CIS-TETRAHYDRO-METHYL-4-METHYLENE-6-PHENYL-2H-PYRAN,
(+−)-CIS-3,6-DIHYDRO-4,6-DIMETHYL-2-PHENYL-2H-PYRAN,
(+−)-CIS-3,6-DIHYDRO-2,4-DIMETHYL-6-PHENYL-2H-PYRAN,
(2E,6Z)-2,6-NONADIENAL,
(2E,6Z)-2,6-NONADIEN-1-OL,
3-PHENYLPROPANAL,
3-(6,6-DIMETHYL-BICYCLO[3.1.1]HEPT-2-EN-2-YL)PROPANAL,
(+−)-2,4-dimethyl-4-phenyltetrahydrofuran,
2,6,6-TRIMETHYL-1,3-CYCLOHEXADIENE-1-CARBALDEHYDE,
(1R,4R)-8-MERCAPTO-3-P-MENTHANONE,
(4E)-4-decenal,
2-ETHOXY-4-METHYLPHENOL,
(+−)-(4Z)-4-cycloocten-1-yl methyl carbonate,
2,6-nonadienenitrile,
(+−)-2-(4-methyl-3-cyclohexen-1-yl)-2-propanethiol, and any combination thereof; and
the group B′ ingredients are selected from the group consisting of:
decanal,
DODECANAL,
1-methoxy-4-[(1E)-1-propen-1-yl]benzene,
4-PHENYL-2-BUTANONE,
(+)-(5S)-5-isopropenyl-2-methyl-2-cyclohexen-1-one,
(+)-(R)-3,7-DIMETHYL-6-OCTENAL,
(+−)-3,7-dimethyl-6-octenal,
5,9-DIMETHYL-4-DECENAL,
2-METHOXY-4-METHYLPHENOL,
cyclopropylmethyl 3-hexenoate,
(+−)-2-pentylcyclopentanone,
(+−)-2,6-DIMETHYL-7-OCTEN-2-OL,
3,7-DIMETHYL-1,6-NONADIEN-3-OL,
ETHYL PHENYLACETATE,
ETHYL 2-METHYL-1,3-DIOXOLANE-2-ACETATE,
3-(2,2-DIMETHYLPROPYL) PYRIDINE,
ETHYL TRICYCLO[5.2.1.0.(2,6)]DECANE-2-CARBOXYLATE,
(+−)-3,7-dimethyl-6-octen-1-yl formate,
(2E)-3,7-dimethyl-2,6-octadien-1-yl formate,
3,7-DIMETHYL-2,6-OCTADIENITRILE,
(+−)-4-HEPTANOLIDE,
1-phenylvinyl acetate,
(8E)-8-undecenal,
10-undecenal,
(9E)-9-undecenal,
(+)-3-[(1R)-4-methyl-3-cyclohexen-1-yl]butanal,
(+−)-3,7-dimethyl-1,6-octadien-3-ol,
METHYL PHENYLACETATE,
(+−)-2-METHYLDECANENITRILE,
2-PHENYLETHYL ACETATE,
2-PHENYLETHANOL,
(+−)-ethyl 2-acetyl-4-methyl-4-pentenoate,
(Z)-3-NONENYL ACETATE,
(Z)-6-NONENYL ACETATE,
(Z,Z)-3,6-NONADIENYL ACETATE,
(2E,6Z)-2,6-NONADIENYL ACETATE,
3-(4,4-dimethyl-1-cyclohexen-1-yl)propanal,
(+−)-3-PHENYLBUTANAL,
2-METHYL-3-HEXANONE OXIME,
1,3-undecadien-5-yne,
(+−)-4-methoxy-2,5-dimethyl-3 (2H)-furanone,
(+−)-6-methoxy-2,6-dimethylheptanal, and any combination thereof.

The fragrance composition according to the present disclosure may further comprise at least 0.1%, typically at least 2%, more typically at least 4%, by weight relative to the total weight of the composition, of at least two ingredients selected from group A″ ingredients, group B″ ingredients, or a combination thereof, each of the at least two ingredients having a volatility greater than or equal to 1000 μg/L, wherein the group A″ ingredients are selected from the group consisting of:
1-(2-PYRIDYL)-1-ETHANONE,
2,3-PENTANEDIONE,
1-(1,3-THIAZOL-2-YL)-1-ETHANONE,
HEPTANAL,
OCTANAL,
(+−)-ETHYL 2-METHYLPENTANOATE,
2-FURANMETHANETHIOL,
(+−)-1-octen-3-ol,
(Z)-6-NONENAL,
4-MERCAPTO-4-METHYL-2-PENTANONE,
(1RS,6RS)-3,6-dimethyl-3-cyclohexene-1-carbaldehyde,
4,6-dimethyl-3-cyclohexene-1-carbaldehyde,
(+−)-ethyl 2-methylbutanoate,
ETHYL BENZOATE,
ETHYL BUTANOATE,
ETHYL HEXANOATE,
3-ETHYL-2,5-DIMETHYLPYRAZINE,
2-ETHYL-3,5-DIMETHYLPYRAZINE,
ETHYL ISOBUTYRATE,
2-ETHYL-3-METHYLPYRAZINE,
5-METHYL-2-HEPTEN-4-ONE,
2-ISOPROPYL-3-METHOXYPYRAZINE,
(+−)-2,6-DIMETHYL-5-HEPTENAL,
2-methoxy-3-methylpyrazine,
2-methoxy-6-methylpyrazine,
2-ISOPROPYL-4-METHYLTHIAZOLE,
DIALLYL DISULFIDE,
(1R,5R)-4,7,7-trimethyl-6-thiabicyclo[3.2.1]oct-3-ene,
(1R,4R,5R)-4,7,7-trimethyl-6-thiabicyclo[3.2.1]octane,
1-decen-4-yne,
PYRAZOBUTYLE,
2-METHOXY-3-(1-METHYLPROPYL)PYRAZINE,
(+−)-methyl 2,2-dimethyl-6-methylidenecyclohexanecarboxylate,
(+−)-2-ETHYL-4,4-DIMETHYL-1,3-OXATHIANE,
(+−)-1-METHOXY-3-HEXANETHIOL,
2,3,5-TRIMETHYLPYRAZINE,
TRIMETHYLAMINE,
(+−)-ETHYL 3-METHYL-2-OXOPENTANOATE,
2,4-dimethyl-3-cyclohexene-1-carbaldehyde, and any combination thereof;
and the group B″ ingredients are selected from the group consisting of:
ACETOPHENONE,
(+−)-3-HYDROXY-2-BUTANONE,
Hexanal,
Nonanal,
3-METHYLBUTYL PROPIONATE,
2-METHYLBUTYL PROPIONATE,
METHYL (2E)-2-METHYL-2-HEXENOATE,
1,1-diethoxy-3,7-dimethyl-2,6-octadiene,
CYCLOHEXYL ACETATE,
DIBUTYL SULFIDE,
1,3,3-trimethyl-2-oxabicyclo[2.2.2]octane,
(+)-(1R,2R,4S)-1,3,3-trimethylbicyclo[2.2.1]heptan-2-ol,
2-PHENYLPROPANAL,
3,5,6-TRIMETHYL-3-CYCLOHEXENE-1-CARBALDEHYDE,
2,4,6-TRIMETHYL-3-CYCLOHEXENE-1-CARBALDEHYDE,
METHYL 3-(METHYLTHIO)PROPANOATE,
1-METHOXY-4-METHYLBENZENE,
7-METHYL-3-METHYLENE-1,6-OCTADIENE,
(3Z)-1-[2-buten-1-yloxy]-3-hexene,
2-methyl-4-propyl-1,3-oxathiane,
(2-METHOXYETHYL)BENZENE,
(Z)-3-HEXENYL ACETATE,
(Z)-3-HEXENYL FORMATE,
4-methyl-2-(2-methyl-1-propen-1-yl)tetrahydro-2H-pyran,
(−)-(2S)-1-oxo-1-(2-propanyloxy)-2-propanyl 2,2-dimethylpropanoate,
(2E)-2-octen-4-one,
(2E)-2-HEXENAL, and any combination thereof.

The fragrance composition may further comprise additional ingredients typically used in the perfumery industry, such as a perfumery carrier.

As used herein, “perfumery carrier” refers to a material which is practically neutral from a perfumery point of view, i.e., that it does not significantly alter the organoleptic properties of perfuming ingredients. Said carrier may be a liquid or a solid. Exemplary liquid carriers include, but are not limited to, an emulsifying system, i.e., a solvent and a surfactant system, or a solvent typically used in perfumery. A detailed description of the nature and type of solvents commonly used in perfumery cannot be exhaustive. However, suitable solvents include, but are not limited to, glycols, such as propylene or butylene glycol; glycerol; dipropyleneglycol and its monoether, 1,2,3-propanetriyl triacetate, dimethyl glutarate, dimethyl adipate, 1,3-diacetyloxypropan-2-yl acetate, diethyl phthalate, isopropyl myristate, rosin resins (such as Abalyn® available from Eastman), benzyl benzoate, benzyl alcohol, 2-(2-ethoxyethoxy)-1-ethanol, tri-ethyl citrate, and mixtures thereof; and naturally derived solvents, such as glycerol and vegetable oils, including palm oil, sunflower oil, linseed oil, and mixtures thereof. In some instances, for example, the perfumery carrier may be ethanol, water/ethanol mixtures, limonene or other terpenes, isoparaffins such as those known under the trademark Isopar® (available from Exxon Chemical) or glycol ethers and glycol ether esters, such as those known under the trademark Dowanol® (available from Dow Chemical Company), or hydrogenated castors oils such as those known under the trademark Cremophor® RH 40 (available from BASF).

Solid carriers are materials to which the fragrance composition or some element of the fragrance composition can be chemically or physically bound. In general, such solid carriers are employed either to stabilize the composition, or to control the rate of evaporation of the compositions or of some ingredients. Solid carriers are of current use in the art and a person skilled in the art knows how to reach the desired effect. Suitable solid carriers include, but are not limited to, absorbing gums or polymers or inorganic materials, such as porous polymers, cyclodextrines, dextrines, maltodextrines wood-based materials, organic or inorganic gels, clays, gypsum talc or zeolites.

Other suitable solid carriers include encapsulating materials. Examples of such materials may comprise wall-forming and plasticizing materials, such as glucose syrups, natural or modified starches, hydrocolloids, cellulose derivatives, polyvinyl acetates, polyvinylalcohols, proteins or pectins, plant gums such as acacia gum (Gum Arabic), urea, sodium chloride, sodium sulphate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, carbohydrates, saccharides such as sucrose, mono-, di-, and polysaccharides and derivatives such as chitosan, starch, cellulose, carboxymethyl methylcellulose, methylcellulose, hydroxyethyl cellulose, ethyl cellulose, propyl cellulose, polyols/sugar alcohols such as sorbitol, maltitol, xylitol, erythritol, and isomalt, polyethylene glycol (PEG), polyvinyl pyrrolidin (PVP), polyvinyl alcohol, acrylamides, acrylates, polyacrylic acid and related, maleic anhydride copolymers, amine-functional polymers, vinyl ethers, styrenes, polystyrenesulfonates, vinyl acids, ethylene glycol-propylene glycol block copolymers, vegetable gums, gum acacia, pectins, xanthanes, alginates, carragenans, citric acid or any water soluble solid acid, fatty alcohols or fatty acids and mixtures thereof.

Other suitable encapsulating materials are described in reference texts known to those of skill in the art, such as H. Scherz, Hydrokolloide: Stabilisatoren, Dickungs-und Geliermittel in Lebensmitteln, Band 2 der Schriftenreihe Lebensmittelchemie, Lebensmittelqualität, Behr's Verlag Gmbh & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation techniques.

Other exemplary solid carriers include core-shell capsules with resins of aminoplast, polyamide, polyester, polyurea or polyurethane type, and mixtures thereof, made using techniques well-known to those of ordinary skill in the art, such as phase separation induced by polymerization, interfacial polymerization, coacervation, or a combination thereof, optionally in the presence of a polymeric stabilizer or of a cationic copolymer.

Resins may be produced by the polycondensation of an aldehyde (e.g., formaldehyde, 2,2-dimethoxyethanal, glyoxal, glyoxylic acid or glycolaldehyde, and mixtures thereof) with an amine such as urea, benzoguanamine, glycoluryl, melamine, methylol melamine, methylated methylol melamine, guanazole and the like, as well as mixtures thereof. Alternatively, one may use preformed resins like alkylolated polyamines such as those commercially available under the trademark Urac® (origin: Cytec Technology Corp.), Cymel® (origin: Cytec Technology Corp.), Urecoll® or Luracoll® (origin: BASF).

Other suitable resins are the those produced by the polycondensation of a polyol, like glycerol, and a polyisocyanate, for example, a trimer of hexamethylene diisocyanate, a trimer of isophorone diisocyanate or xylylene diisocyanate or a Biuret of hexamethylene diisocyanate or a trimer of xylylene diisocyanate with trimethylolpropane (marketed as Takenate® by Mitsui Chemicals), among which a trimer of xylylene diisocyanate with trimethylolpropane and a Biuret of hexamethylene diisocyanate are of mention.

The encapsulation of perfumes by polycondensation of amino resins, namely melamine-based resins with aldehydes is well-known in the art. Pertinent publications include, but are not limited to, K. Dietrich et al. Acta Polymerica, 1989, vol. 40, pages 243, 325 and 683, as well as 1990, vol. 41, page 91 and U.S. Pat. No. 4,396,670 issued Aug. 2, 1983. The general knowledge in encapsulation technology is very significant and cannot be exhaustive. More recent publications of pertinence, which disclose suitable uses of such microcapsules, are represented, for example, by the article of K. Bruyninckx and M. Dusselier, ACS Sustainable Chemistry & Engineering, 2019, vol. 7, pages 8041-8054. These publications are incorporated herein by reference.

The fragrance composition may optionally comprise at least one perfumery adjuvant.

The at least one perfumery adjuvant is an ingredient capable of imparting an additional added benefit such as a color, a particular light resistance, chemical stability, etc. A detailed description of the nature and type of adjuvant commonly used in perfuming compositions cannot be exhaustive, but it has to be mentioned that said ingredients are well known to a person skilled in the art.

Exemplary perfumery adjuvants include, but are not limited to, viscosity agents (e.g., surfactants, thickeners, gelling and/or rheology modifiers), stabilizing agents (e.g., preservatives, antioxidant, heat/light and or buffers or chelating agents, such as BHT), coloring agents (e.g., dyes and/or pigments), preservatives (e.g. antibacterial or antimicrobial or antifungal or anti irritant agents), abrasives, skin cooling agents, fixatives, insect repellants, ointments, vitamins and mixtures thereof.

Fixatives, also known as “modulators”, are agents having the capacity to affect the manner in which the odor, and in particular the evaporation rate and intensity, of the compositions incorporating said modulator can be perceived by an observer or user thereof, over time, as compared to the same perception in the absence of the modulator. In particular, the modulator allows prolonging the time during which their fragrance is perceived. Suitable modulators include, but are not limited to, methyl glucoside polyol; ethyl glucoside polyol; propyl glucoside polyol; isocetyl alcohol; PPG-3 myristyl ether; neopentyl glycol diethylhexanoate; sucrose laurate; sucrose dilaurate, sucrose myristate, sucrose palmitate, sucrose stearate, sucrose distearate, sucrose tristearate, hyaluronic acid disaccharide sodium salt, sodium hyaluronate, propylene glycol propyl ether; dicetyl ether; polyglycerin-4 ethers; isoceteth-5; isoceteth-7, isoceteth-10; isoceteth-12; isoceteth-15; isoceteth-20; isoceteth-25; isoceteth-30; disodium lauroamphodipropionate; hexaethylene glycol monododecyl ether; and their mixtures; neopentyl glycol diisononanoate; cetearyl ethylhexanoate; panthenol ethyl ether, DL-panthenol, N-hexadecyl n-nonanoate, noctadecyl n-nonanoate, a profragrance, cyclodextrin, an encapsulation, and any combination thereof. In an embodiment, the fragrance further comprises a fixative.

The fragrance compositions described herein may further comprise one or more other odorant ingredients so long as the presence and/or concentrations of the said odorant ingredient or ingredients do not alter the long-lasting trail performance of the composition. Such odorant ingredients include, but are not limited to, (+−)-1-(3-CYCLOOCTEN-1-YL)-1-PROPANOL, ETHYL CYCLOHEXANECARBOXYLATE, 7-(3-METHYLBUTYL)-2H-1,5-BENZODIOXEPIN-3(4H)-ONE, METHYL (E)-4,7-OCTADIENOATE, 11-OXA-16-HEXADECANOLIDE, (2E,5Z)-5,6,7-TRIMETHYL-2,5-OCTADIEN-4-ONE, 3-METHYL-5-CYCLOTETRADECEN-1-ONE, (+−)-2-TERT-BUTYL-5-METHYL-2-PROPYL-2,5-DIHYDROFURAN, (+−)-(Z)-1-(3-CYCLOOCTEN-1-YL)ETHANONE, 2-ETHYL-N-METHYL-N-(3-METHYLPHENYL)BUTANAMIDE, (3Z)-1-METHYL-3-HEXENYL SALICYLATE, (+−)-6-METHOXY-2,6-DIMETHYLOCTANAL, cyclohexylidene(2-methylphenyl)acetonitrile, (+−)-2-(2,2,7,7-tetramethyltricyclo[6.2.1.0˜1,6˜]undec-5-en-5-yl)-1-propanol, (+−)-4-vinyl-1-cyclohexene-1-carbaldehyde, (+−)-2-[(3,5-dimethyl-3-hexen-2-yl)oxy]-2-methylpropyl cyclopropanecarboxylate, (+−)-1-methoxy-7,9-dimethyl-2-oxaspiro[5.5]undec-8-ene, (+−)-1-ethoxy-7,9-dimethyl-2-oxaspiro[5.5]undec-8-ene, (3S,5R,8S)-5-isopropenyl-3,8-dimethyl-1,2,3,4,5,6,7,8-octahydro-1-azulenol, (+−)-8-(2-butanyl)-5,6,7,8-tetrahydroquinoline, (4Z)-9-hydroxy-5,9-dimethyl-4-decenal, (+−)-2-[(1E)-3-ethoxy-1-buten-1-yl]-1,3,3-trimethylcyclohexene, 3-(4-isobutyl-2-methylphenyl)propanal, (+−)-[1-methyl-2-(5-methyl-4-hexen-2-yl)cyclopropyl]methanol, (+−)-2,4,7-trimethyl-6-octen-1-ol, METHYL 2-METHYLBENZOATE, (+−)-METHYL 3-METHYL-1-CYCLOHEXANECARBOXYLATE, (+−)-1-(2,4-DIMETHYL-3-CYCLOHEXEN-1-YL)-1-PROPANOL, oxacycloheptadecan-2-one, ((1RS,2RS)-2,4-dimethylcyclohex-3-enyl)methyl acetate, (+)-(1S)-1-[(1R)-3,3-dimethylcyclohexyl]ethyl ethyl propanedioate, (+)-(1S)-1-[(1R)-3,3-dimethylcyclohexyl]ethyl ethyl propanedioate, (+−)-4-METHYL-2-PROPYL-1,3-OXATHIANE, ETHYL 3-(1-CYCLOHEXEN-1-YL)PROPANOATE, (+)-(1R,4S,9S,10R,13S)-5,5,9,13-tetramethyl-14,16-dioxatetracyclo[11.2.1.0˜1,10˜0.0˜4,9˜]hexadecane, 2,5-DIMETHYL-5-PHENYL-1-HEXEN-3-ONE, (3Z)-3-HEXENYL CYCLOPROPANECARBOXYLATE, (Z)-6-DECENAL, (1RS,2SR,6SR,7RS,8RS)-tricyclo[5.2.1.0(2,6)]dec-8-ylacetaldehyde, 3Aa-ETHYL-PERHYDRO-6,6,9Aa-TRIMETHYL-5AbH,9BaH-NAPHTHO[2,1-B]FURAN, 3-(4-ISOPROPYL-1-PHENYL)PROPANAL, (+−)-3-ETHYL-PERHYDRO-BENZO[B]FURAN-2-ONE, 5-METHYL-5-PHENYL-3-HEXANONE, (+−)-CIS-2-ETHYL-4-METHYL-1,3-OXATHIANE, (+−)-1-ETHYL-2-(3-METHYLBUTYL)-1-CYCLOPENTANOL, 3-(trans-4-isobutylcyclohexyl)propanal, METHYL 2,4-DIHYDROXY-3-METHYLBENZOATE, 1,1-DIMETHYL-2-PHENYLETHYL ISOBUTYRATE, (+−)-2-methoxy-4-(4-methyl-3,6-dihydro-2H-pyran-2-yl) phenol, (+−)-2,2,6,6,7,8,8-heptamethyl-4,5,6,7,8,8b-hexahydro-3aH-indeno[4,5-d][1,3]dioxole, (+−)-4-(HEPTYLOXY)-3-METHYLBUTANAL, (3E)-4-methyl-3-decen-5-one, (+−)-2-(5-isopropyl-2-methyltetrahydrothiophen-2-yl) ethanol, (+−)-ethyl 4-methyl-2-oxocyclohexanecarboxylate, (1RS,2RS,4RS)-ethyl bicyclo[2.2.1]hept-5-ene-2-carboxylate, (+−)-4,6-dimethyl-2-(1-phenylethyl)-3,6-dihydro-2H-pyran, 2-methyl-1-(5,5,6-trimethylbicyclo[2.2.1]hept-2-yl)-2-propanol, tricyclo[5.2.1.0˜2,6˜]dec-3-en-8-yl butyrate, (+−)-7,7,8,9,9-pentamethyl-6,7,8,9-tetrahydro-5H-cyclopenta[h]quinazoline, (6aRS,9aRS)-7,7,8,9,9-pentamethyl-6,6a,7,8,9,9a-hexahydro-5H-cyclopenta[h]quinazoline, 5,7,8-trimethyltricyclo[5.2.1.0˜2,6˜]decan-8-ol, (+−)-2-methyl-3-pentanyl (2E)-2-butenoate, 3-methyl-1-benzofuran-5-ol, (−)-4,6,6,7,8,8-hexamethyl-1,3,4,6,7,8-hexahydrocyclopenta[g]isochromene, (+−)-3a-isopropyl-2,6-dimethyloctahydro-1-benzofuran, 5-(hexyloxy)-2,2-dimethyltetrahydrofuran, (+−)-4-ethylidene-2-propoxycyclohexanol, 3-[(1R,5S)-6,6-dimethylbicyclo[3.1.1]hept-2-en-2-yl]-2,2-dimethylpropanenitrile, (+−)-(1E)-1-(4,6-dimethyl-3-cyclohexen-1-yl)-2-methyl-1-penten-3-one, (+−)-(3E)-3-(methoxymethylene)tricyclo[5.2.1.0˜2,6˜]decane, (2E,5E)-2-methoxy-6-methyl-2,5-octadiene, (14S)-14-methyloxacyclohexadec-10-en-2-one, 5-propyltetrahydro-2H-pyran-2-one, (2Z)-(2,4,4-trimethylcyclopentylidene)acetonitrile, 6-cyclopentylidenehexanal, (4Z)-6-(2,2,3-trimethyl-3-cyclopenten-1-yl)-4-hexenal, (+−)-(2Z)-3,5,7-trimethyl-2-octenenitrile, 3,5,7-trimethyloctanenitrile, 6-(2,4,4-trimethylcyclopentylidene) hexanal, (1SR,6RS,8SR)-2,2-dimethyl-8-propyltricyclo[6.2.1.0˜1,6˜]undecan-7-one, 2,6-dimethyl-4-(2,2,3-trimethylcyclopent-3-en-1-yl)cyclohex-2-en-1-ol, 5-(6-hepten-1-yl)dihydro-2(3H)-furanone, (+−)-(1Z,5E)-9-methoxy-2,5,10-trimethyl-1,5-cyclododecadiene, 2-(3,3-dimethyl-1-cyclohexen-1-yl)-2,5,5-trimethyl-1,3-dioxane, 8-CYCLOHEXADECEN-1-ONE, 4-DECYLPYRIDINE, (+−)-8EXO-ETHOXY-EXO-TRICYCLO[5.2.1.0(2,6)]DECANE, (+−)-ethyl tetrahydro-2-furancarboxylate, 3-METHYL-4-DECANOLIDE, (Z)-4-ETHYL-3-OCTENENITRILE, (+)-3-[(4R)-4-isopropyl-1-cyclohexen-1-yl]propanal, bicyclo[2.2.1]hept-5-en-2-yl acetate, (+−)-ethyl 2-cyclopenten-1-ylacetate, (+−)-2-METHYLDODECANAL, 4-(3-METHYLBUTYL) CYCLOHEXANOL, 6-methyl-4-(2,2,3-trimethyl-3-cyclopenten-1-yl)-2-cyclohexen-1-ol, 2-((1RS,2SR)-2-ethylcyclohexyloxy) acetaldehyde, (+−)-5-cyclohexyl-2-methyl-1-pentanol, [(cis-4-methylcyclohexyl)oxy]acetaldehyde, {[cis-4-(2-propanyl)cyclohexyl]oxy}acetaldehyde, {[trans-4-(2-methyl-2-propanyl)cyclohexyl]oxy}acetaldehyde, (1R,3S,5R,7R,8R,10S,13R)-5,7,9,9,13-pentamethyl-5-propyl-4,6-dioxatetracyclo[6.5.1.0˜1,10˜0.0˜3,7˜]tetradecane, (+−)-2-methyl-5-propyl-2-cyclohexen-1-one, 3,4-dimethylbenzonitrile3,4-dimethylbenzonitrile, diethyl (2E)-2-methyl-2-butenedioate, 5-MERCAPTO-5-METHYL-3-HEXANONE, (+−)-3-(4-ISOBUTYLPHENYL)-2-METHYLPROPANAL, (+−)-2,5,5-trimethyl-1,2,3,4,4a,5,6,7-octahydro-2-naphthalenol, (+−)-ethyl 3-mercapto-2-methylbutanoate, (−)-(3S,6E)-3,7,11-trimethyl-6,10-dodecadienal, (+)-(1S,2R)-1,2,3,3-TETRAMETHYL-BICYCLO[2.2.1]HEPTAN-2-OL (1S,4R)-1,2,3,3-TETRAMETHYL-BICYCLO[2.2.1]HEPTAN-2-OL, 3-methyl-5-[(1R)-2,2,3-trimethylcyclopentyl]-2-pentanone, 2-ethoxy-4-(hydroxymethyl) phenol, (+−)-3-cyclohexen-1-ylmethyl 2-hydroxypropanoate, 2-methoxy-2-methyl-heptane, allyl (1R,6S)-2,2,6-trimethylcyclohexanecarboxylate, (+−)-6-hydroxy-2,6-dimethylheptanal, 3-methyl-2-pentyl-2-cyclopenten-1-one, 2,2-dimethyl-3-(3-methylphenyl)-1-propanol, BELLANONE, CANTALOOP, CEYLANIL, CYCLAMENTIOL, DIVINIRIS, EDENFLOR, FLOWERPOOL, and the like.

The trail performance, typically long-lasting trail performance, may be measured using methods and instrumentation known to those of ordinary skill in the art. Exemplary methods and instrumentation are described in US Patent Publication No. 20190128782 (Luetkenhaus, et al.), U.S. Pat. No. 6,067,842 (Gygax, et al.), and US Patent Publication No. 20090320559 (Lemieuvre, et al.), the contents of these references being incorporated herein by reference. One suitable method, which is preferred, is described in PCT Application No. PCT/EP2022/052718, filed Feb. 4, 2022, the contents of which is incorporated herein by reference.

The fragrance compositions according to the present disclosure may be prepared according to any method known to those of ordinary skill in the art. The ordinarily-skilled artisan is perfectly able to design optimal formulations for the desired effect by admixing the above mentioned components to arrive at the desired composition by applying standard knowledge and concepts known to those of ordinary skill and by utilizing routine optimization methodologies.

In the second aspect, the present disclosure relates to a perfumed consumer product comprising the fragrance composition described herein.

The form of the consumer product is not particularly limited. In an embodiment, the perfumed consumer product is a perfume, a fabric care product, a body-care product, a cosmetic preparation, a skin-care product, an air care product, or a home care product.

In another embodiment, the perfumed consumer product is a fine perfume, a splash, eau de parfum, a cologne, a shave or after-shave lotion, a liquid or solid or unit-dose detergent, a fabric softener, a solid or liquid fabric scent-booster, a fabric refresher, an ironing water, a paper, a bleach, a carpet cleaners, curtain-care products, a shampoo, a leave-on or rinse-off hair conditioner, a coloring preparation, a color care product, a hair shaping product, a dental care product, a disinfectant, an intimate care product, a hair spray, a vanishing cream, a deodorant or antiperspirant, hair remover, tanning or sun product, a nail product, a skin cleansing, a makeup, a perfumed soap, a shower or bath mousse, oil or gel, or a foot/hand care products, a hygiene product, an air freshener, a “ready to use” powdered air freshener, a mold remover, a furniture care product, a wipe, a dish detergent or hard-surface detergent, a leather care product, or a car care product.

In an embodiment, the perfumed consumer product comprises the fragrance composition in an amount from 1% to 95%, typically from 2% to 80%, more typically from 3% to 70%, by weight relative to the total weight of the perfumed consumer product.

In an embodiment, the perfumed consumer product comprises the fragrance composition in an amount from 1% to 30%, typically from 2% to 20%, more typically from 3% to 10%, by weight relative to the total weight of the perfumed consumer product.

In another embodiment, the at least two group A ingredients are present in an amount of from 0.01% to 95%, typically from 0.02% to 50%, more typically from 0.03% to 20%, by weight relative to the total weight of the perfumed consumer product.

In an embodiment, the at least two group A ingredients are present in an amount of from 0.1% to 15%, typically from 0.2% to 10%, more typically from 0.3% to 5%, by weight relative to the total weight of the perfumed consumer product.

In an embodiment, the at least two group B ingredients are present in an amount of from 0.01% to 95%, typically from 0.02% to 50%, more typically from 0.03% to 20%, by weight relative to the total weight of the perfumed consumer product.

In an embodiment, the at least two group B ingredients are present in an amount of from 0.1% to 15%, typically from 0.2% to 10%, more typically from 0.3% to 5%, by weight relative to the total weight of the perfumed consumer product.

In the third aspect, the present disclosure relates to the use of the fragrance composition or the perfumed consumer product described herein to provide a long-lasting fragrance trail.

The compositions, products, and uses, according to the present disclosure are further illustrated by the following non-limiting examples.

### Example 1. Trail Performance of Inventive Floral Composition

Two fragrance compositions, one inventive composition (“FLORTRAIL High”) and one comparative composition (“FLORTRAIL Low”) were evaluated by a panel of 10 experts. The compositions are summarized below in Table 1 and 2 below. Amounts are expressed as percent by weight and volatility is designated as follows—I: volatility>1000 μg/L; II: volatility from 1000 μg/L to 100 μg/L; III: volatility from 100 μg/L to 10 μg/L; IV: volatility from 10 μg/L to 0.001 μg/L.

TABLE 1
FLORTRAIL LOW

| | Amount | | |
|---|---|---|---|
| Component | (%) | Group | Volatility |
| benzyl acetate | 0.67 | — | II |
| ETHYL 2,6,6-TRIMETHYL-1,3- | 0.11 | — | II |
| CYCLOHEXADIENE-1-CARBOXYLATE | | | |
| (Z)-3,7-DIMETHYL-2,6-OCTADIEN-1-OL | 2.01 | — | II |
| (+−)-ALPHA-TERPINEOL | 1.34 | — | II |
| (+−)-3,7-DIMETHYL-3-OCTANOL | 5.35 | — | II |
| (E)-3,7-DIMETHYL-2,6-OCTADIENYL | 1.67 | — | III |
| ACETATE | | | |
| tricyclo[5.2.1.0~2,6~]dec-3-en-8-yl | 1.00 | — | III |
| acetate (A) + tricyclo[5.2.1.0~2,6~]dec- | | | |
| 4-en-8-yl acetate (B) | | | |
| 2-METHYL-4-PHENYL-2-BUTANOL | 1.67 | — | III |
| (+−)-3,7-DIMETHYL-6-OCTEN-1-OL | 2.01 | — | III |
| 4-CYCLOHEXYL-2-METHYL-2-BUTANOL | 11.15 | — | III |
| (+−)-TETRAHYDRO-2-ISOBUTYL-4-METHYL- | 16.72 | — | III |
| 4(2H)-PYRANOL | | | |
| (+−)-7-hydroxy-3,7-dimethyloctanal | 5.02 | — | III |
| [trans-4-(2-propanyl)cyclohexyl]methanol (A) + | 2.79 | — | III |
| [cis-4-(2-propanyl)cyclohexyl]methanol (B) | | | |
| (2E)-2-benzylideneoctanal | 6.69 | — | IV |
| methyl 2-((1RS,2RS)-3-oxo-2- | 31.77 | — | IV |
| pentylcyclopentyl)acetate | | | |
| (2E)-2-METHYL-3-(4-METHYLPHENYL)-2- | 1.00 | — | IV |
| PROPEN-1-OL | | | |
| CYCLOHEXYLIDENE(PHENYL)ACETONITRILE | 1.67 | — | IV |
| (3Z)-3-hexen-1-yl salicylate | 6.69 | — | IV |
| 2-PHENYLETHYL 2-HYDROXYBENZOATE | 0.67 | — | IV |

TABLE 2
FLORTRAIL High

| | Amount | | |
|---|---|---|---|
| Component | (%) | Group | Volatility |
| ETHYL BENZOATE | 0.6 | A | I |
| (Z)-3,7-DIMETHYL-1,6-NONADIEN-3-OL + (E)- | 10 | B | II |
| 3,7-DIMETHYL-1,6-NONADIEN-3-OL | | | |
| (−)-(2S)-1-oxo-1-(2-propanyloxy)-2- | 3 | B | I |
| propanyl 2,2-dimethylpropanoate | | | |
| 2-PHENYLETHANOL | 3 | B | II |
| ETHYL 4,6,6-TRIMETHYL-1,3- | 0.2 | A | II |
| CYCLOHEXADIENE-1-CARBOXYLATE | | | |
| (+−)-3,7-dimethyl-1,6-octadien-3-ol | 20 | B | II |
| (E)-3,7-DIMETHYL-2,6-OCTADIEN-1-OL | 3.6 | B | III |
| (+−)-(3E)-3-methyl-4-(2,6,6-trimethyl-2- | 4 | B | III |
| cyclohexen-1-yl)-3-buten-2-one + (+−)- | | | |
| (1E)-1-(2,6,6-trimethyl-2-cyclohexen-1-yl)- | | | |
| 1-penten-3-one | | | |
| (+−)-2-methyl-3-[4-(2-methyl-2- | 15 | A | III |
| propanyl)phenyl]propanal | | | |
| (+−)-2,4-dimethyl-4,4a,5,9b- | 6 | A | III |
| tetrahydroindeno[1,2-d][1,3]dioxine | | | |
| (ISOMER A) + (+−)-2,4-dimethyl-4,4a,5,9b- | | | |
| tetrahydroindeno[1,2-d][1,3]dioxine | | | |
| (ISOMER B) | | | |
| BENZYL (E)-2-METHYL-2-BUTENOATE | 0.2 | B | III |
| (2RS,4SR)-4-methyl-6-phenyl-2-hexanol + | 4 | B | III |
| (2RS,4RS)-4-methyl-6-phenyl-2-hexanol | | | |
| TRICYCLO[5.2.1.0(2,6)]DEC-3-EN-8-YL | 1.8 | B | III |
| PROPANOATE + TRICYCLO[5.2.1.0(2,6)]DEC- | | | |
| 4-EN-8-YL PROPANOATE | | | |
| methyl [(1RS,2RS)-3-oxo-2- | 22 | — | IV |
| pentylcyclopentyl]acetate (A) + methyl | | | |
| [(1RS,2SR)-3-oxo-2-pentylcyclopentyl]acetate | | | |
| (B) | | | |
| (4E)-4-methyl-5-(4-methylphenyl)-4-pentenal | 2 | B | IV |
| (+−)-3-methyl-5-phenyl-1-pentanol | 4.6 | B | IV |

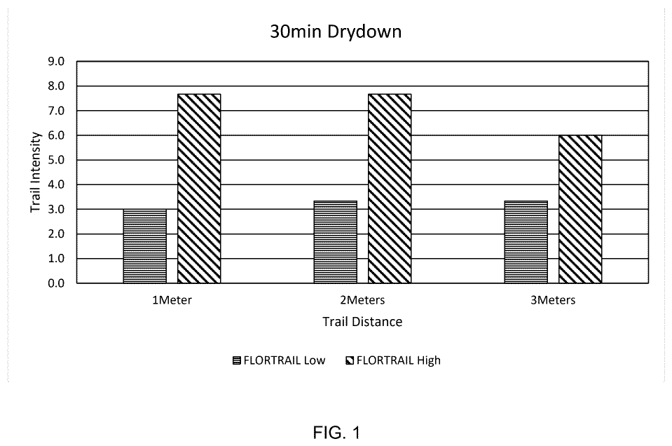

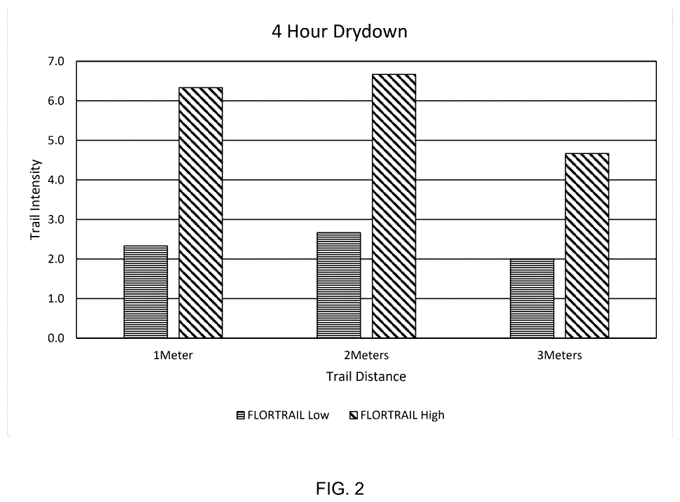

The panel assessed the performance of the perfume trail at various distances (1 meter, 2 meters, and 3 meters). The device described in PCT Application No. PCT/EP2022/052718, filed Feb. 4, 2022, was used to evaluate trail at various times and distances from a finite source. The results are shown in FIGS. 1 and 2. FIG. 1 shows the trail intensity of each of the compositions evaluated after a 30-minute drydown at 1 meter, 2 meters, and 3 meters. FIG. 2 shows the trail intensity of each of the compositions evaluated after a 4-hour drydown at 1 meter, 2 meters, and 3 meters.

As shown in FIGS. 1 and 2, the trail intensity of FLORTRAIL High according to the present disclosure was higher than that of the comparative FLORTRAIL Low after 30-minute and 4-hour drydown at 1 meter, 2 meters, and 3 meters.

### Example 2. Trail Performance of Inventive Fruity Composition

Two fragrance compositions, one inventive composition (“FRUITYTRAIL High”) and one comparative composition (“FRUITYTRAIL Low”) were evaluated according to the method described in Example 1. The compositions are summarized below in Table 3 and 4 below. Amounts are expressed as percent by weight and volatility is designated as follows—I: volatility>1000 μg/L; II: volatility from 1000 μg/L to 100 μg/L; III: volatility from 100 μg/L to 10 μg/L; IV: volatility from 10 μg/L to 0.001 μg/L.

TABLE 3
FRUITYTRAIL Low

| | Amount | | |
|---|---|---|---|
| Component | (%) | Group | Volatility |
| (+−)-ISOPROPYL 2- | 1.35 | — | I |
| METHYLBUTANOATE | | | |
| ETHYL 2,6,6-TRIMETHYL-1,3- | 3.36 | — | II |
| CYCLOHEXADIENE-1-CARBOXYLATE | | | |
| (+−)-(2E)-1-(2,2-dimethyl-6- | 1.68 | — | II |
| methylenecyclohexyl)-2-buten-1-one | | | |
| (2RS,4SR,6RS)-2,4,6-trimethyl-4-phenyl- | 8.41 | — | II |
| 1,3-dioxane + (2RS,4RS,6RS)-2,4,6- | | | |
| trimethyl-4-phenyl-1,3-dioxane | | | |
| (Z)-3-NONENYL ACETATE + (Z)-6- | 0.10 | B | II |
| NONENYL ACETATE + (Z,Z)-3,6- | | | |
| NONADIENYL ACETATE + (2E,6Z)-2,6- | | | |
| NONADIENYL ACETATE | | | |
| (−)-propyl (2S)-2-[(2-methyl-2- | 6.73 | — | II |
| butanyl)oxy]propanoate | | | |
| (+−)-3,7-DIMETHYL-3-OCTANOL | 13.45 | — | II |
| 1,1-DIMETHYL-2-PHENYLETHYL | 1.68 | — | III |
| BUTANOATE | | | |
| 4-CYCLOHEXYL-2-METHYL-2-BUTANOL | 13.45 | — | III |
| ALLYL 3-CYCLOHEXYLPROPANOATE | 6.73 | — | III |
| diethyl cis-1,4-cyclohexanedicarboxylate + | 10.09 | — | III |
| diethyl trans-1,4-cyclohexanedicarboxylate | | | |
| 2-phenoxyethyl 2-methylpropanoate | 19.17 | — | III |
| (+−)-(E)-4-METHYL-3-DECEN-5-OL | 4.37 | — | III |
| (+−)-4-DECANOLIDE | 2.69 | B | IV |
| (+−)-(Z)-7-DECEN-4-OLIDE | 2.02 | — | IV |
| methyl {(1RS,2RS)-3-oxo-2-[(2Z)-2- | 3.36 | — | IV |
| penten-1-yl]cyclopentyl}acetate | | | |
| 4-(1,3-BENZODIOXOL-5-YL)-2- | 1.35 | — | IV |
| BUTANONE | | | |

TABLE 4
FRUITYTRAIL High

| | Amount | | |
|---|---|---|---|
| Component | (%) | Group | Volatility |
| (+−)-ETHYL 2-METHYLPENTANOATE | 0.59 | A | I |
| (3Z)-hex-3-en-1-yl methyl carbonate | 0.88 | — | I |
| (+−)-2,6-DIMETHYL-5-HEPTENAL | 0.88 | A | I |
| (+−)-ethyl 2-methylbutanoate | 0.59 | A | I |
| (2RS,4SR)-2-methyl-4-propyl-1,3- | 4.41 | B | I |
| oxathiane + (2RS,4RS)-2-methyl-4- | | | |
| propyl-1,3-oxathiane | | | |
| (−)-(2S)-1-oxo-1-(2-propanyloxy)-2- | 1.47 | B | I |
| propanyl 2,2-dimethylpropanoate | | | |
| (2E)-2-octen-4-one | 0.88 | B | I |
| (+−)-6-pentyltetrahydro-2H-pyran-2-one | 2.35 | A | II |
| (Z)-3,7-DIMETHYL-1,6-NONADIEN-3- | 14.71 | B | II |
| OL + (E)-3,7-DIMETHYL-1,6- | | | |
| NONADIEN-3-OL | | | |
| ETHYL 2-METHYL-1,3-DIOXOLANE-2- | 8.82 | B | II |
| ACETATE | | | |
| ALLYL HEPTANOATE | 5.88 | — | II |
| (+−)-3,7-dimethyl-1,6-octadien-3-ol | 24.12 | B | II |
| (Z)-3-NONENYL ACETATE + (Z)-6- | 0.88 | B | II |
| NONENYL ACETATE + (Z,Z)-3,6- | | | |
| NONADIENYL ACETATE + (2E,6Z)-2,6- | | | |
| NONADIENYL ACETATE | | | |
| ETHYL 4,6,6-TRIMETHYL-1,3- | 0.29 | A | II |
| CYCLOHEXADIENE-1-CARBOXYLATE | | | |
| (+−)-(2E)-1-(2,6,6-trimethyl-2- | 2.94 | A | III |
| cyclohexen-1-yl)-2-buten-1-one | | | |
| (+−)-4-NONANOLIDE | 1.18 | A | III |
| (3S,3aS,6R,7aR)-3,6- | 0.88 | A | III |
| dimethylhexahydro-1-benzofuran- | | | |
| 2(3H)-one | | | |
| 4-(4-hydroxyphenyl)-2-butanone | 11.76 | A | IV |
| methyl [(1RS,2RS)-3-oxo-2- | 14.71 | B | IV |
| pentylcyclopentyl]acetate + methyl | | | |
| [(1RS,2SR)-3-oxo-2- | | | |
| pentylcyclopentyl]acetate | | | |
| (+−)-6-[(2Z)-2-penten-1- | 1.76 | B | IV |
| yl]tetrahydro-2H-pyran-2-one | | | |

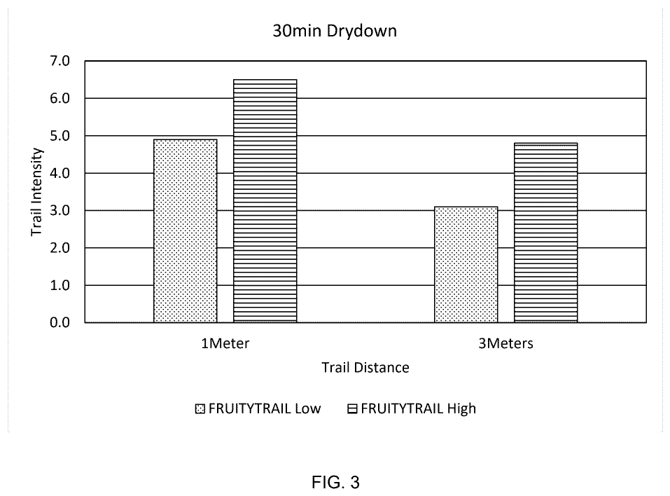

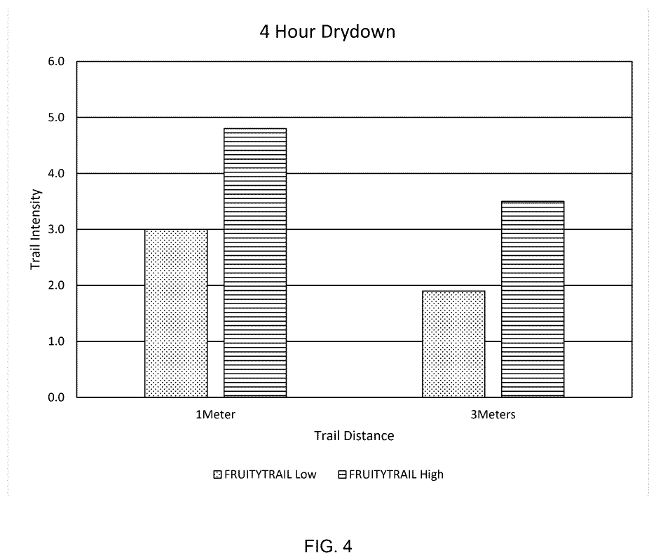

The panel assessed the performance of the perfume trail at various distances (1 meter and 3 meters). The device described in PCT Application No. PCT/EP2022/052718, filed Feb. 4, 2022, was used to evaluate trail at various times and distances from a finite source. The results are shown in FIGS. 3 and 4. FIG. 3 shows the trail intensity of each of the compositions evaluated after a 30-minute drydown at 1 meter and 3 meters. FIG. 4 shows the trail intensity of each of the compositions evaluated after a 4-hour drydown at 1 meter and 3 meters.

As shown in FIGS. 3 and 4, the trail intensity for “FRUITYTRAIL High” prepared in accordance with the present disclosure is higher than that of the comparative “FRUITYTRAIL Low” formulation after 30-minute and 4-hour drydown at 1 and 3 meters.

### Example 3. Trail Performance of Inventive Composition Versus Commercial Product

An inventive product (“Product A”) was constructed from a floral chassis composition and a fragrance composition that was designed to impart high intensity trail impression at various concentrations when added to the floral chassis. The components of the floral chassis are summarized in Table 5 below. The components of the fragrance composition are summarized in Table 6 below. Amounts are expressed as weight percent and volatility is designated as follows—I: volatility>1000 μg/L; II: volatility from 1000 μg/L to 100 μg/L; III: volatility from 100 μg/L to 10 μg/L; IV: volatility from 10 μg/L to 0.001 μg/L.

TABLE 5
Floral chassis

| | Amount | | |
|---|---|---|---|
| Component | (wt %) | Group | Volatility |
| (2E)-2-benzylideneoctanal | 15 | — | 4 |
| methyl 2-((1RS,2RS)-3-oxo-2- | 15 | — | 4 |
| pentylcyclopentyl)acetate | | | |
| (+−)-7-hydroxy-3,7-dimethyloctanal | 15 | — | 3 |
| [trans-4-(2-propanyl)cyclohexyl]methanol + | 10 | — | 3 |
| [cis-4-(2-propanyl)cyclohexyl]methanol | | | |
| (Z)-3,7-DIMETHYL-2,6-OCTADIEN-1-OL | 10 | — | 2 |
| 2-phenoxyethanol | 10 | — | 3 |
| (+−)-3,7-DIMETHYL-6-OCTEN-1-OL | 5 | — | 3 |
| (+−)-TETRAHYDRO-2-ISOBUTYL-4- | 5 | — | 3 |
| METHYL-4(2H)-PYRANOL | | | |
| methyl {(1RS,2RS)-3-oxo-2-[(2Z)-2- | 5 | — | 4 |
| penten-1-yl]cyclopentyl}acetate | | | |
| (+−)-3,7-dimethyl-1,6-octadien-3-ol | 5 | B | 2 |
| (+−)-ALPHA-TERPINEOL | 5 | — | 2 |
| Total | 100 | | |

TABLE 6
Fragrance composition

| | Amount | | |
|---|---|---|---|
| Component | (wt %) | Group | Volatility |
| (+−)-2,4-dimethyl-4,4a,5,9b- | 25 | A | 3 |
| tetrahydroindeno[1,2-d][1,3]dioxine | | | |
| (ISOMER A) + (+−)-2,4-dimethyl-4,4a,5,9b- | | | |
| tetrahydroindeno[1,2-d][1,3]dioxine | | | |
| (ISOMER B) | | | |
| 2-METHOXYNAPHTHALENE | 20 | A | 3 |
| 1-(3-METHYL-1-BENZOFURAN-2- | 15 | A | 3 |
| YL)ETHANONE | | | |
| (+−)-(2,5-dimethyl-2,3-dihydro- | 10 | A | 4 |
| 1H-inden-2-yl)methanol | | | |
| (+−)-(2E)-1-(2,6,6-trimethyl-2- | 5 | A | 3 |
| cyclohexen-1-yl)-2-buten-1-one | | | |
| 3-(3,3-dimethyl-2,3-dihydro-1H-inden-5- | 5 | A | 4 |
| yl)propanal + 3-(1,1-dimethyl-2,3-dihydro- | | | |
| 1H-inden-4-yl)propanal + 3-(1,1-dimethyl- | | | |
| 2,3-dihydro-1H-inden-5-yl)propanal | | | |
| 2-methoxy-4-[(1E)-1-propen-1-yl]phenol | 5 | A | 3 |
| (1′S,3′R)-{1-METHYL-2-[(1′,2′,2′- | 5 | A | 4 |
| TRIMETHYLBICYCLO[3.1.0]HEX-3′- | | | |
| YL)METHYL]CYCLOPROPYL}METHANOL | | | |
| (4E)-4-methyl-5-(4-methylphenyl)-4- | 5 | B | 4 |
| pentenal | | | |
| (+−)-4-methylene-2-phenyltetrahydro-2H- | 4 | A | 3 |
| pyran + (+−)-4-methyl-6-phenyl-3,6-dihydro- | | | |
| 2H-pyran + (+−)-4-methyl-2-phenyl-3,6- | | | |
| dihydro-2H-pyran | | | |
| (+−)-1-(5-propyl-1,3-benzodioxol-2- | 1 | A | 3 |
| yl)ethanone | | | |
| Total | 100 | | |

Product A was then created by adding 6 parts of the fragrance composition to 94 parts of the floral chassis. The resulting product comprised 4.50% group A ingredients with volatility III, 1.00% group A ingredients with volatility IV, and 0.05% group B ingredients with volatility IV.

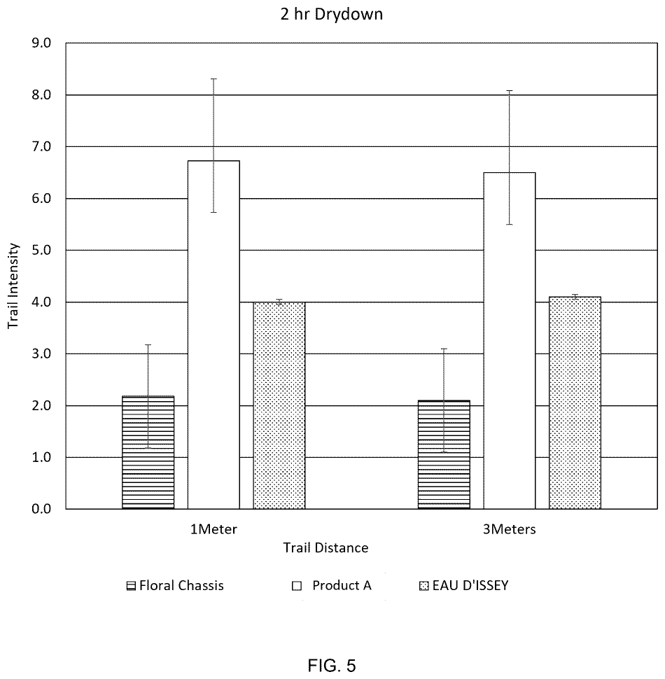

A panel of 11 respondents was used to evaluate Product A and a commercial product, EAU D'ISSEY by ISSEY MIYAKE. The fragrance EAU D'ISSEY was used as a benchmark for trail performance of above average for floral fragrances. The device described in PCT Application No. PCT/EP2022/052718 was used to reproduce trail conditions at distances of 1 m and 3 m at 2 hours. The results are summarized in FIG. 5. FIG. 5 shows the trail intensity of the floral chassis, Product A, and EAU D'ISSEY after a 2-hour drydown at 1 meter and 3 meters.

The disclosed subject matter has been described with reference to specific details of particular embodiments thereof. It is not intended that such details be regarded as limitations upon the scope of the disclosed subject matter except insofar as and to the extent that they are included in the accompanying claims.

Therefore, the exemplary embodiments described herein are well adapted to attain the ends and advantages mentioned as well as those that are inherent therein. The particular embodiments disclosed above are illustrative only, as the exemplary embodiments described herein may be modified and practiced in different but equivalent manners apparent to those of ordinary skill in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the exemplary embodiments described herein. The exemplary embodiments described herein illustratively disclosed herein suitably may be practiced in the absence of any element that is not specifically disclosed herein and/or any optional element disclosed herein.

What is claimed is:

1. A fragrance composition comprising at least 4%, typically at least 5%, more typically at least 6%, by weight relative to the total weight of the composition, of at least two group A ingredients, wherein the group A ingredients are selected from the group consisting of:
(3aRS,5aRS,8aRS,8bSR)-2,2,6,6,7,8,8-heptamethyldecahydro-2H-indeno[4,5-b]furan,
4-methoxybenzaldehyde,
(+−)-1-(5-propyl-1,3-benzodioxol-2-yl)ethenone,
3-(4-TERT-BUTYLPHENYL)PROPANAL,
3-BUTYLIDENE-1-BENZO[C]FURANONE,
7-methyl-2H-1,5-benzodioxepin-3(4H)-one,
5-ISOPROPYL-2-METHYLPHENOL,
7-ISOPROPYL-2H,4H-1,5-BENZODIOXEPIN-3-ONE,
4,8-cyclododecadien-1-one,
(E)-3-PHENYL-2-PROPENENITRILE,
3-methyl-5-phenyl-2-pentenenitrile,
(3S,3aR,5R,8S,8aS)-5-isopropenyl-3,8-dimethyloctahydro-3a(1H)-azulenol,
isopropenyl-1,4-dimethyldecahydro-4-azulenol,
(1R,3R,6S,7S,8S)-2,2,6,8-tetramethyltricyclo[5.3.1.0˜3,8˜]undecan-3-ol,
(1R,3R,6S,7S,8S)-3-ethoxy-2,2,6,8-tetramethyltricyclo[5.3.1.0˜3,8˜]undecane,
(+−)-6-METHYL-7-OXA-1-THIA-4-AZASPIRO[4.4]NONANE,
4-METHYLPHENYL 3-METHYLBUTANOATE,
(2E)-1-(2,6,6-trimethyl-1,3-cyclohexadien-1-yl)-2-buten-1-one,
(+−)-(2E)-1-(2,6,6-trimethyl-2-cyclohexen-1-yl)-2-buten-1-one,
(2E)-2-dodecenal,
(+−)-4-methylene-2-phenyltetrahydro-2H-pyran,
(+−)-4-methyl-6-phenyl-3,6-dihydro-2H-pyran,
(+−)-4-methyl-2-phenyl-3,6-dihydro-2H-pyran,
(E)-3-METHYL-5-(2,2,3-TRIMETHYL-3-CYCLOPENTEN-1-YL)-4-PENTEN-2-OL,
ETHYL 2,3-EPOXY-3-PHENYLBUTANOATE,
2-ETHYL-3-HYDROXY-4 (4H)-PYRANONE,
2-methoxy-4-(2-propen-1-yl) phenol,
4-allyl-2-methoxyphenyl acetate,
2-methyl-4-[(1R)-2,2,3-trimethyl-3-cyclopenten-1-yl]-4-penten-1-ol,
(+−)-5-ethyl-4-hydroxy-2-methyl-3 (2H)-furanone,
(+−)-2-ethyl-4-hydroxy-5-methyl-3 (2H)-furanone,
4-METHYLPHENOXY)ACETALDEHYDE,
2-methoxy-4-[(1E)-1-propen-1-yl]phenol,
8-isopropylquinoline,
6-isopropylquinoline,
3,7-DIMETHYL-6-OCTEN-1-OL,
3,7-DIMETHYL-7-OCTEN-1-OL,
(+−)-2-methyl-3-[4-(2-methyl-2-propanyl)phenyl]propanal,
(+−)-2,4-dimethyl-4,4a,5,9b-tetrahydroindeno[1,2-d][1,3]dioxine,
3,12-TRIDECADIENENITRILE,
methyl 2-aminobenzoate,
2,6-DIMETHOXYPHENOL,
3,6-dimethylhexahydro-1-benzofuran-2(3H)-one,
1-(5,5-dimethyl-1-cyclohexen-1-yl)-4-penten-1-one,
(+−)-1-(5-ethyl-5-methyl-1-cyclohexen-1-yl)-4-penten-1-one,
1-(3-METHYL-1-BENZOFURAN-2-YL)ETHANONE,
5-NONANOLIDE,
(+−)-4-NONANOLIDE,
PERHYDRO-2-CHROMENONE,
1-(2-AMINOPHENYL)-1-ETHANONE,
(−)-(1R,3R,6S,7S,8S)-2,2,6,8-tetramethyltricyclo[5.3.1.0˜3,8˜]undecan-3-ol,
(E)-2-ETHOXY-5-(1-PROPENYL)PHENOL,
3-METHYLINDOLE,
1-oxaspiro[4.5]decan-2-one,
(4Z)-4-dodecenal,
1,5,9-TRIMETHYL-4,8-DECADIENYL ACETATE,
3-METHYL-4-OCTANOLIDE,
3-PROPYLPHENOL,
2-METHOXYNAPHTHALENE;
(+−)-2-[4-(2-hydroxy-2-methylpropyl)phenyl]propanal,
3-(3-(2-hydroxy-2-methylpropyl)phenyl)propanal,
3-[4-(2-hydroxy-2-methylpropyl)phenyl]propanal,
4-(4-methoxyphenyl)-2-butanone,
2-(3-phenylpropyl)pyridine,
(−)-(2E)-2-ethyl-4-[(1R)-2,2,3-trimethyl-3-cyclopenten-1-yl]-2-buten-1-ol,
(+−)-6-pentyltetrahydro-2H-pyran-2-one,
3-(3,3-dimethyl-2,3-dihydro-1H-inden-5-yl)propanal
3-(1,1-dimethyl-2,3-dihydro-1H-inden-4-yl)propanal
3-(1,1-dimethyl-2,3-dihydro-1H-inden-5-yl)propanal,
(E)-2-METHOXY-4-(1-PROPENYL)PHENYL ACETATE,
(1'S,3′R)-{1-METHYL-2-[(1′,2′,2′-TRIMETHYLBICYCLO[3.1.0]HEX-3′-YL)METHYL]CYCLOPROPYL}METHANOL,
(+−)-(2,5-dimethyl-2,3-dihydro-1H-inden-2-yl)methanol;
(+−)-3-(4-hydroxy-4-methylpentyl)-3-cyclohexene-1-carbaldehyde,
(+−)-4-(4-hydroxy-4-methylpentyl)-3-cyclohexene-1-carbaldehyde,
METHYL 2,4-DIHYDROXY-3,6-DIMETHYLBENZOATE,
4-(4-hydroxyphenyl)-2-butanone,
(+)-(1R,7R)-10,10-DIMETHYL-TRICYCLO[7.1.1.0(2,7)]UNDEC-2-EN-4-ONE,
(+−)-5-heptyldihydro-2(3H)-furanone,
4-hydroxy-3-methoxybenzaldehyde,
(−)-(3aR,5aS,9aS,9bR)-3a,6,6,9a-tetramethyldodecahydronaphtho[2,1-b]furan,
(−)-(1R,3S,7R,8R,10S,13R)-5,5,7,9,9,13-HEXAMETHYL-4,6-DIOXATETRACYCLO[6.5.1.0(1,10).0(3,7)]TETRADECANE, and any combination thereof;
and wherein each of the at least two group A ingredients have a volatility of less than 100 μg/L, typically from 100 μg/L to 10 μg/L or from 10 μg/L to 0.001 μg/L.

2. The fragrance composition according to claim 1, wherein the at least two group A ingredients are present in an amount of from 4% to 95%, typically from 5% to 80%, more typically from 6% to 70%, by weight relative to the total weight of the composition.

3. The fragrance composition according to claim 1 or 2, further comprising at least 1%, typically at least 3%, more typically at least 4%, by weight relative to the total weight of the composition, of at least two group B ingredients, wherein the group B ingredients are selected from the group consisting of:
1-(4-METHOXYPHENYL)-1-ETHANONE,
(+−)-8alpha, 8A-EPOXY-PERHYDRO-2beta,5,5-TRIMETHYL-2-TRANS-NAPHTHALENOL,
(+−)-4A,8A-EPOXY-PERHYDRO-2,5,5-TRIMETHYL-2-NAPHTHALENOL,
(+−)-4-(C-2,3-EPOXY-2,6,6-TRIMETHYL-R-1-CYCLOHEXYL)-2-BUTANONE,
4-METHOXYBENZYL ACETATE,
4-METHOXY-1-BENZENEMETHANOL,
BENZYL (E)-2-METHYL-2-BUTENOATE,
(+−)-1,1,2,3,3-pentamethyl-1,2,3,5,6,7-hexahydro-4H-inden-4-one,
cedr-8-ene,
1-(cedr-8-en-9-yl)ethenone,
(2E)-3-phenyl-2-propen-1-ol,
(E)-3-PHENYL-2-PROPENAL,
(E)-3-PHENYL-2-PROPENYL ACETATE,
3-methyl-2-[(2Z)-2-penten-1-yl]-2-cyclopenten-1-one,
3-hydroxy-2-methyl-4H-pyran-4-one,
ALLYL (CYCLOHEXYLOXY)ACETATE,
2-methoxy-4-propylphenol,
4-methyl-2-phenyltetrahydro-2H-pyran,
(2E)-1-(2,6,6-trimethyl-1-cyclohexen-1-yl)-2-buten-1-one,
ETHYL (E)-3-PHENYL-2-PROPENOATE,
3-(4-ETHYLPHENYL)-2,2-DIMETHYLPROPANAL,
3-(2-ETHYLPHENYL)-2,2-DIMETHYLPROPANAL,
(+−)-3-(3-ISOPROPYL-1-PHENYL) BUTANAL,
(+−)-(E)-3-METHYL-4-(2,6,6-TRIMETHYL-2-CYCLOHEXEN-1-YL)-3-BUTEN-2-ONE,
(E)-1-(2,6,6-TRIMETHYL-2-CYCLOHEXEN-1-YL)-1-PENTEN-3-ONE,
(+−)-(E)-1-(2,2-DIMETHYL-6-METHYLENE-1-CYCLOHEXYL)-1-PENTEN-3-ONE,
(E)-1-(2,6,6-TRIMETHYL-1-CYCLOHEXEN-1-YL)-1-PENTEN-3-ONE,
(E)-3,7-DIMETHYL-2,6-OCTADIEN-1-OL,
benzo[d][1,3]dioxole-5-carbaldehyde,
(+−)-(3E)-3-methyl-4-(2,6,6-trimethyl-2-cyclohexen-1-yl)-3-buten-2-one,
(+−)-(1E)-1-(2,6,6-trimethyl-2-cyclohexen-1-yl)-1-penten-3-one,
(+−)-(E)-TRANS-alpha-IRONE,
(+−)-(E)-CIS-alpha-IRONE,
(+−)-(E)-beta-IRONE,
(+−)-(3E)-3-methyl-4-(2,6,6-trimethyl-2-cyclohexen-1-yl)-3-buten-2-one,
(+−)-(1E)-1-(2,6,6-trimethyl-2-cyclohexen-1-yl)-1-penten-3-one,
3,7-dimethyl-2,6-nonadienenitrile,
3,7-dimethyl-3,6-nonadienenitrile,
10-METHYL-alpha-IONONE,
10-METHYL-beta-IONONE,
10-METHYL-GAMMA-IONONE,
(1E)-1-(2,6,6-trimethyl-1-cyclohexen-1-yl)-1-penten-3-one,
(+)-3,3-dimethyl-5-[(1S)-2,2,3-trimethyl-3-cyclopenten-1-yl]-4-penten-2-ol,
(+−)-6-propyltetrahydro-2H-pyran-2-one,
(1RS,4aSR,8RS,8aSR)-2,2,6,8-tetramethyl-1,2,3,4,4a,5,8,8a-octahydro-1-naphthalenol,
(3Z)-3-hexen-1-yl hexanoate,
2,4,6,8-TETRAMETHYLNONYL ACETATE,
(−)-3,3-dimethyl-5-[(1R)-2,2,3-trimethyl-3-cyclopenten-1-yl]-4-penten-2-ol,
(2RS,4aRS,8aSR)-5,5,8a-trimethyldecahydro-2-naphthalenyl acetate,
(2S,5R)-5-methyl-2-(2-propanyl)cyclohexanone oxime,
2,6,10,10-tetramethyl-1-oxaspiro[4.5]decan-6-ol,
(4Z)-4-DODECENENITRILE,
DODECANENITRILE,
Thymol,
TRICYCLO[5.2.1.0(2,6)]DEC-3-EN-8-YL PROPANOATE,
TRICYCLO[5.2.1.0(2,6)]DEC-4-EN-8-YL PROPANOATE,
2-TERT-BUTYL-1,4-DIMETHOXYBENZENE,
(+−)-(3E)-4-(2,6,6-trimethyl-2-cyclohexen-1-yl)-3-buten-2-one,
(3E)-4-(2,6,6-trimethyl-1-cyclohexen-1-yl)-3-buten-2-one,
4-methyl-6-phenyl-2-hexanol,
9-hydroxy-5,9-dimethyl-4-decenal;
7-PROPYL-2H,4H-1,5-BENZODIOXEPIN-3-ONE,
ALLYL PHENOXYACETATE,
(3aRS,5aSR,9aSR,9bRS)-3a,6,6,9a-tetramethyldodecahydronaphtho[2,1-b]furan,
(3aRS,5aSR,9aSR,9bSR)-3a,6,6,9a-tetramethyldodecahydronaphtho[2,1-b]furan,
(3aRS,5aSR,9aSR,9bRS)-3a,6,6,9a-tetramethyldodecahydronaphtho[2,1-b]furan,
(3aRS,5aSR,9aSR,9bSR)-3a,6,6,9a-tetramethyldodecahydronaphtho[2,1-b]furan,
(+−)-2-ETHYL-4-(2,2,3-TRIMETHYL-3-CYCLOPENTEN-1-YL)-2-BUTEN-1-OL,
(E)-3-PHENYL-2-PROPENYL PROPANOATE,
methyl [3-oxo-2-pentylcyclopentyl]acetate,
2-CHROMENONE,
(+−)-4-DECANOLIDE,
(+−)-5-octyldihydro-2(3H)-furanone,
(+−)-6-heptyltetrahydro-2H-pyran-2-one,
(+−)-3-(1,3-BENZODIOXOL-5-YL)-2-METHYLPROPANAL,
3-(3,3-dimethyl-2,3-dihydro-1H-inden-5-yl)propanal,
3-(1,1-dimethyl-2,3-dihydro-1H-inden-5-yl)propanal,
4-formyl-2-methoxyphenyl 2-methylpropanoate,
(+−)-8-sec-butylquinoline,
(+−)-6-sec-butylquinoline,
(+−)-6-[(2Z)-2-penten-1-yl]tetrahydro-2H-pyran-2-one,
2-ethoxy-4-(methoxymethyl) phenol,
1-(2-naphthyl)ethenone,
(4E)-4-methyl-5-(4-methylphenyl)-4-pentenal,
2-{2-[(1R)-4-methyl-3-cyclohexen-1-yl]propyl}cyclopentanone,
(+)-(4R,4aS,6R)-4,4a-dimethyl-6-(1-propen-2-yl)-4,4a,5,6,7,8-hexahydro-2(3H)-naphthalenone,
1-[2,2,6-trimethylcyclohexyl]-3-hexanol,
2-tridecenenitrile,
3-tridecenenitrile,
4-METHYLPHENYL PHENYLACETATE,
(+−)-3-methyl-5-phenyl-1-pentanol,
(+−)-2,3,3-TRIMETHYL-1-INDANONE,
2-(4-METHYL-1,3-THIAZOL-5-YL)-1-ETHANOL,
7-(2-methyl-2-propanyl)-2H-1,5-benzodioxepin-3(4H)-one,
(E)-2-TRIDECENAL,
6-hexyltetrahydro-2H-pyran-2-one,
2-methoxy-4-methylphenyl methyl carbonate,
(+)-(1S,2S,3S,5R)-2,6,6-trimethylspiro[bicyclo[3.1.1]heptane-3,1′-cyclohexane]-2′-en-4′-one,
(1S,4S,9S,10R,13R)-5,5,9,13-tetramethyl-14,16-dioxatetracyclo[11.2.1.0˜1,10˜0.0˜4,9˜]hexadecane,
(1R,4S,9S,10R,13S)-5,5,9,13-tetramethyl-14,16-dioxatetracyclo[11.2.1.0˜1,10˜0.0˜4,9˜]hexadecane, and any combination thereof;
and wherein each of the at least two group B ingredients have a volatility of less than 100 μg/L, typically from 100 μg/L to 10 μg/L or from 10 μg/L to 0.001 μg/L.

4. The fragrance composition according to claim 3, wherein the at least two group B ingredients are present in an amount of from 1% to 95%, typically from 3% to 80%, more typically from 4% to 70%, by weight relative to the total weight of the composition.

5. The fragrance composition according to claim 3 or 4, wherein at least one of the at least two group A ingredients and/or at least one of the at least two group B ingredients have a volatility of from 10 μg/L to 0.001 μg/L.

6. The fragrance composition according to any one of claims 3-5, wherein at least one of the at least two group A ingredients and/or at least one of the at least two group B ingredients have a volatility of from 100 μg/L to 10 μg/L.

7. The fragrance composition according to any one of claims 1-6, further comprising at least 0.1%, typically at least 2%, more typically at least 4%, by weight relative to the total weight of the composition, of at least two ingredients selected from group A′ ingredients, group B′ ingredients, or a combination thereof, each of the at least two ingredients having a volatility in the range of from 1000 μg/L to 100 μg/L, wherein the group A′ ingredients are selected from the group consisting of:
1-(PYRAZINYL)-1-ETHANONE,
(+−)-2-methylundecanal,
10-undecenal,
9-undecenal,
ALLYL (3-METHYLBUTOXY)ACETATE,
(+−)-ALLYL (2-METHYLBUTOXY)ACETATE,
(−)-(5R)-5-isopropenyl-2-methyl-2-cyclohexen-1-one,
(+−)-3-mercaptohexyl acetate,
(Z)-4-DECENAL,
(2E)-1-[(1RS,2SR)-2,6,6-trimethyl-3-cyclohexen-1-yl]-2-buten-1-one,
4-ETHYL-2-METHOXYPHENOL,
ETHYL 4,6,6-TRIMETHYL-1,3-CYCLOHEXADIENE-1-CARBOXYLATE,
(+−)-4-HYDROXY-2,5-DIMETHYL-3 (2H)-FURANONE,
(3E,5Z)-1,3,5-UNDECATRIENE,
(+−)-PERHYDRO-4alpha, 8Abeta-DIMETHYL-4A-NAPHTHALENOL,
2-METHOXYPHENOL,
8-ISOPROPYL-6-METHYL-BICYCLO[2.2.2]OCT-5-ENE-2-CARBALDEHYDE,
2-HEXYL-2-CYCLOPENTEN-1-ONE,
2-METHOXY-3-(4-METHYLPENTYL)PYRAZINE,
METHYL 2-OCTYNOATE,
1-(4-methylphenyl)ethenone,
(+−)-2-methyldecanal,
(2E,6Z)-1,1-DIETHOXY-2,6-NONADIENE,
(Z)-2-NONENAL,
2-HYDROXY-3-METHYL-2-CYCLOPENTEN-1-ONE,
GAMMA OCTALACTONE,
2,6,6-TRIMETHYL-2-CYCLOHEXENE-1,4-DIONE,
4-METHYLPHENOL,
4-METHYLPHENYL ACETATE,
4-METHYLPHENYL ISOBUTYRATE,
(+−)-CIS-TETRAHYDRO-METHYL-4-METHYLENE-6-PHENYL-2H-PYRAN,
(+−)-CIS-3,6-DIHYDRO-4,6-DIMETHYL-2-PHENYL-2H-PYRAN,
(+−)-CIS-3,6-DIHYDRO-2,4-DIMETHYL-6-PHENYL-2H-PYRAN,
(2E,6Z)-2,6-NONADIENAL,
(2E,6Z)-2,6-NONADIEN-1-OL,
3-PHENYLPROPANAL,
3-(6,6-DIMETHYL-BICYCLO[3.1.1]HEPT-2-EN-2-YL)PROPANAL,
(+−)-2,4-dimethyl-4-phenyltetrahydrofuran,
2,6,6-TRIMETHYL-1,3-CYCLOHEXADIENE-1-CARBALDEHYDE,
(1R,4R)-8-MERCAPTO-3-P-MENTHANONE,
(4E)-4-decenal,
2-ETHOXY-4-METHYLPHENOL,
(+−)-(4Z)-4-cycloocten-1-yl methyl carbonate,
2,6-nonadienenitrile,
(+−)-2-(4-methyl-3-cyclohexen-1-yl)-2-propanethiol,
and any combination thereof;
and the group B′ ingredients are selected from the group consisting of:
decanal,
DODECANAL,
1-methoxy-4-[(1E)-1-propen-1-yl]benzene,
4-PHENYL-2-BUTANONE,
(+)-(5S)-5-isopropenyl-2-methyl-2-cyclohexen-1-one,
(+)-(R)-3,7-DIMETHYL-6-OCTENAL,
(+−)-3,7-dimethyl-6-octenal,
5,9-DIMETHYL-4-DECENAL,
2-METHOXY-4-METHYLPHENOL,
cyclopropylmethyl 3-hexenoate,
(+−)-2-pentylcyclopentanone,
(+−)-2,6-DIMETHYL-7-OCTEN-2-OL,
3,7-DIMETHYL-1,6-NONADIEN-3-OL,
ETHYL PHENYLACETATE,
ETHYL 2-METHYL-1,3-DIOXOLANE-2-ACETATE,
3-(2,2-DIMETHYLPROPYL) PYRIDINE,
ETHYL TRICYCLO[5.2.1.0.(2,6)]DECANE-2-CARBOXYLATE,
(+−)-3,7-dimethyl-6-octen-1-yl formate,
(2E)-3,7-dimethyl-2,6-octadien-1-yl formate,
3,7-DIMETHYL-2,6-OCTADIENITRILE,
(+−)-4-HEPTANOLIDE,
1-phenylvinyl acetate,
(8E)-8-undecenal,
10-undecenal,
(9E)-9-undecenal,
(+)-3-[(1R)-4-methyl-3-cyclohexen-1-yl]butanal,
(+−)-3,7-dimethyl-1,6-octadien-3-ol,
METHYL PHENYLACETATE,
(+−)-2-METHYLDECANENITRILE,
2-PHENYLETHYL ACETATE,
2-PHENYLETHANOL,
(+−)-ethyl 2-acetyl-4-methyl-4-pentenoate,
(Z)-3-NONENYL ACETATE,
(Z)-6-NONENYL ACETATE,
(Z,Z)-3,6-NONADIENYL ACETATE,
(2E,6Z)-2,6-NONADIENYL ACETATE,
3-(4,4-dimethyl-1-cyclohexen-1-yl)propanal,
(+−)-3-PHENYLBUTANAL,
2-METHYL-3-HEXANONE OXIME,
1,3-undecadien-5-yne,
(+−)-4-methoxy-2,5-dimethyl-3 (2H)-furanone,
(+−)-6-methoxy-2,6-dimethylheptanal,
and any combination thereof.

8. The fragrance composition according to any one of claims 1-7, wherein at least one of the at least two group A ingredients is selected from the group consisting of:
(+−)-2-[4-(2-hydroxy-2-methylpropyl)phenyl]propanal,
3-(3-(2-hydroxy-2-methylpropyl)phenyl)propanal,
3-[4-(2-hydroxy-2-methylpropyl)phenyl]propanal,
(+−)-1-(5-propyl-1,3-benzodioxol-2-yl)ethenone,
(3S,3aR,5R,8S,8aS)-5-isopropenyl-3,8-dimethyloctahydro-3a(1H)-azulenol,
7-isopropenyl-1,4-dimethyldecahydro-4-azulenol,
(1R,3R,6S,7S,8S)-2,2,6,8-tetramethyltricyclo[5.3.1.0˜3,8˜]undecan-3-ol,
(1R,3R,6S,7S,8S)-3-ethoxy-2,2,6,8-tetramethyltricyclo[5.3.1.0˜3,8˜]undecane,
(+−)-6-METHYL-7-OXA-1-THIA-4-AZASPIRO[4.4]NONANE,
(2E)-2-dodecenal,
(+−)-4-methylene-2-phenyltetrahydro-2H-pyran,
(+−)-4-methyl-6-phenyl-3,6-dihydro-2H-pyran,
(+−)-4-methyl-2-phenyl-3,6-dihydro-2H-pyran,
3,6-dimethylhexahydro-1-benzofuran-2(3H)-one,
(+−)-1-(5-ethyl-5-methyl-1-cyclohexen-1-yl)-4-penten-1-one,
(−)-(1R,3R,6S,7S,8S)-2,2,6,8-tetramethyltricyclo[5.3.1.0˜3,8˜]undecan-3-ol,
1-oxaspiro[4.5]decan-2-one,
(4Z)-4-dodecenal,
3-PROPYLPHENOL, and any combination thereof.

9. The fragrance composition according to any one of claims 4-6, wherein at least one of the at least two group B ingredients is selected from the group consisting of:
7-PROPYL-2H,4H-1,5-BENZODIOXEPIN-3-ONE,
(4E)-4-methyl-5-(4-methylphenyl)-4-pentenal,
7-(2-methyl-2-propanyl)-2H-1,5-benzodioxepin-3(4H)-one,
2-methoxy-4-methylphenyl methyl carbonate,
(+)-(1S,2S,3S,5R)-2,6,6-trimethylspiro[bicyclo[3.1.1]heptane-3,1′-cyclohexane]-2′-en-4′-one,
(1S,4S,9S,10R,13R)-5,5,9,13-tetramethyl-14,16-dioxatetracyclo[11.2.1.0˜1,10˜0.0˜4,9˜]hexadecane,
(1R,4S,9S,10R,13S)-5,5,9,13-tetramethyl-14,16-dioxatetracyclo[11.2.1.0˜1,10˜0.0˜4,9˜]hexadecane,
(+)-3,3-dimethyl-5-[(1S)-2,2,3-trimethyl-3-cyclopenten-1-yl]-4-penten-2-ol,
(1RS,4aSR,8RS,8aSR)-2,2,6,8-tetramethyl-1,2,3,4,4a,5,8,8a-octahydro-1-naphthalenol,
(2RS,4aRS,8aSR)-5,5,8a-trimethyldecahydro-2-naphthalenyl acetate,
(2S,5R)-5-methyl-2-(2-propanyl)cyclohexanone oxime,
2,6,10,10-tetramethyl-1-oxaspiro[4.5]decan-6-ol,
4-methyl-6-phenyl-2-hexanol, and any combination thereof.

10. The fragrance composition according to any one of claims 1-9, further comprising at least 0.1%, typically at least 2%, more typically at least 4%, by weight relative to the total weight of the composition, of at least two ingredients selected from group A″ ingredients, group B″ ingredients, or a combination thereof, each of the at least two ingredients having a volatility greater than or equal to 1000 μg/L, wherein the group A″ ingredients are selected from the group consisting of:
1-(2-PYRIDYL)-1-ETHANONE,
2,3-PENTANEDIONE,
1-(1,3-THIAZOL-2-YL)-1-ETHANONE,
HEPTANAL,
OCTANAL,
(+−)-ETHYL 2-METHYLPENTANOATE,
2-FURANMETHANETHIOL,
(+−)-1-octen-3-ol,
(Z)-6-NONENAL,
4-MERCAPTO-4-METHYL-2-PENTANONE,
(1RS,6RS)-3,6-dimethyl-3-cyclohexene-1-carbaldehyde,
4,6-dimethyl-3-cyclohexene-1-carbaldehyde,
(+−)-ethyl 2-methylbutanoate,
ETHYL BENZOATE,
ETHYL BUTANOATE,
ETHYL HEXANOATE,
3-ETHYL-2,5-DIMETHYLPYRAZINE,
2-ETHYL-3,5-DIMETHYLPYRAZINE,
ETHYL ISOBUTYRATE,
2-ETHYL-3-METHYLPYRAZINE,
5-METHYL-2-HEPTEN-4-ONE,
2-ISOPROPYL-3-METHOXYPYRAZINE,
(+−)-2,6-DIMETHYL-5-HEPTENAL,
2-methoxy-3-methylpyrazine,
2-methoxy-6-methylpyrazine,
2-ISOPROPYL-4-METHYLTHIAZOLE,
DIALLYL DISULFIDE,
(1R,5R)-4,7,7-trimethyl-6-thiabicyclo[3.2.1]oct-3-ene,
(1R,4R,5R)-4,7,7-trimethyl-6-thiabicyclo[3.2.1]octane,
1-decen-4-yne,
PYRAZOBUTYLE,
2-METHOXY-3-(1-METHYLPROPYL)PYRAZINE,
(+−)-methyl 2,2-dimethyl-6-methylidenecyclohexanecarboxylate,
(+−)-2-ETHYL-4,4-DIMETHYL-1,3-OXATHIANE,
(+−)-1-METHOXY-3-HEXANETHIOL,
2,3,5-TRIMETHYLPYRAZINE,
TRIMETHYLAMINE,
(+−)-ETHYL 3-METHYL-2-OXOPENTANOATE,
2,4-dimethyl-3-cyclohexene-1-carbaldehyde, and any combination thereof;
and the group B″ ingredients are selected from the group consisting of:
ACETOPHENONE,
(+−)-3-HYDROXY-2-BUTANONE,
Hexanal,
Nonanal,
3-METHYLBUTYL PROPIONATE,
2-METHYLBUTYL PROPIONATE,
METHYL (2E)-2-METHYL-2-HEXENOATE,
1,1-diethoxy-3,7-dimethyl-2,6-octadiene,
CYCLOHEXYL ACETATE,
DIBUTYL SULFIDE,
1,3,3-trimethyl-2-oxabicyclo[2.2.2]octane,
(+)-(1R,2R,4S)-1,3,3-trimethylbicyclo[2.2.1]heptan-2-ol,
2-PHENYLPROPANAL,
3,5,6-TRIMETHYL-3-CYCLOHEXENE-1-CARBALDEHYDE,
2,4,6-TRIMETHYL-3-CYCLOHEXENE-1-CARBALDEHYDE,
METHYL 3-(METHYLTHIO)PROPANOATE,
1-METHOXY-4-METHYLBENZENE,
7-METHYL-3-METHYLENE-1,6-OCTADIENE,
(3Z)-1-[2-buten-1-yloxy]-3-hexene,
2-methyl-4-propyl-1,3-oxathiane,
(2-METHOXYETHYL)BENZENE,
(Z)-3-HEXENYL ACETATE,
(Z)-3-HEXENYL FORMATE,
4-methyl-2-(2-methyl-1-propen-1-yl)tetrahydro-2H-pyran,
(−)-(2S)-1-oxo-1-(2-propanyloxy)-2-propanyl 2,2-dimethylpropanoate,
(2E)-2-octen-4-one,
(2E)-2-HEXENAL, and any combination thereof.

11. The fragrance composition according to any one of claims 1-10, further comprising a fixative.

12. A perfumed consumer product comprising the fragrance composition according to any one of claims 1-11.

13. The perfumed consumer product according to claim 12, wherein the perfumery consumer product is a perfume, a fabric care product, a body-care product, a cosmetic preparation, a skin-care product, an air care product, or a home care product.

14. The perfumed consumer product according to claim 12 or 13, wherein the perfumery consumer product is a fine perfume, a splash, eau de parfum, a cologne, a shave or after-shave lotion, a liquid or solid or unit-dose detergent, a fabric softener, a solid or liquid fabric scent-booster, a fabric refresher, an ironing water, a paper, a bleach, a carpet cleaners, curtain-care products, a shampoo, a leave-on or rinse-off hair conditioner, a coloring preparation, a color care product, a hair shaping product, a dental care product, a disinfectant, an intimate care product, a hair spray, a vanishing cream, a deodorant or antiperspirant, hair remover, tanning or sun product, a nail product, a skin cleansing, a makeup, a perfumed soap, a shower or bath mousse, oil or gel, or a foot/hand care products, a hygiene product, an air freshener, a “ready to use” powdered air freshener, a mold remover, a furniture care product, a wipe, a dish detergent or hard-surface detergent, a leather care product, or a car care product.

15. Use of the fragrance composition according to any one of claims 1-11 or the perfumed consumer product according to claim 13 or 14 to provide a long-lasting fragrance trail.
